(19) United States
(12) Patent Application Publication
(10) Pub. No.: US 2025/0020518 A1
(43) Pub. Date: Jan. 16, 2025

(54) DIGITAL MICROFLUIDICS (DMF) SYSTEM, CARTRIDGE, AND METHODS FOR THERMAL CALIBRATION OF INTEGRATED HEATERS AND SENSORS

(71) Applicant: Baebies, Inc., Durham, NC (US)

(72) Inventor: Gregory F. Smith, Durham, NC (US)

(73) Assignee: Baebies, Inc., Durham, NC (US)

(21) Appl. No.: 18/715,986

(22) PCT Filed: Dec. 5, 2022
(86) PCT No.: PCT/US2022/051797

§ 371 Date: Jun. 3, 2024

Related U.S. Application Data
(60) Provisional application No. 63/285,798, filed on Dec. 3, 2021.

(51) Int. Cl. G01K 7/18; G01K 15/00

(52) U.S. Cl. CPC G01K 7/18; G01K 15/005

(57) ABSTRACT

Described is thermal control in digital microfluidics (DMF) cartridges and more particularly to a DMF system, cartridge, and methods for thermal calibration of integrated heaters and sensors. In some embodiments, the presently disclosed subject matter provides a DMF system, cartridge, and methods for thermal calibration of integrated heaters and sensors. The presently disclosed DMF system provides a DMF cartridge (or device) having integrated heating. In some embodiments, the presently disclosed DMF system, cartridge, and methods may provide PCB-based sensors for monitoring the temperature at respective PCB-based heaters and wherein the PCB-based sensors may be, for example, copper sense traces of a PCB substrate. In some embodiments, the presently disclosed DMF system, cartridge, and methods may provide thermal calibration software and/or thermal control electronics for performing a thermal calibration process of a selected DMF cartridge.

### CROSS-REFERENCE TO RELATED APPLICATIONS

This application claims the benefit of U.S. Provisional Application No. 63/285,798, filed Dec. 3, 2021, which is incorporated by reference herein in its entirety.

### BACKGROUND

Digital microfluidics (DMF) systems, devices, and/or cartridges are used to process biological materials. In these DMF processes, thermocycling of the biological materials being processed may occur. For example, droplets of the biological materials may be cycled between two processing temperatures. This thermocycling may occur at a certain rate or frequency.

Currently, DMF systems, devices, and/or cartridges use resistance temperature detectors (RTDs) as one method for monitoring and/or controlling the temperature of, for example, a DMF cartridge. An RTD is a passive sensor device whose resistance changes as its temperature changes. For example, the resistance increases as the temperature of the sensor increases. The resistance vs. temperature relationship is well known and is repeatable over time. In some cases, RTDs are high resistance (e.g., 100-1000 ohms) and the accuracy is maintained via control of fabrication techniques and variables. If an RTD is 100-1000 ohms, then a sufficiently small absolute resistance difference from nominal is substantially inconsequential. For example, a standard 100 Ohm platinum RTD changes in resistance by 0.385 Ohms per degree Celsius, so a 0.038 Ohm inaccuracy may represent just a 0.1-degree Celsius error and be relatively inconsequential in many applications.

By contrast, some DMF systems, devices, and/or cartridges may use printed circuit board (PCB)-based heaters and PCB-based thermal sensors. The PCB-based sensors may use, for example, small copper traces as the sensing mechanism. Generally, the resistance of these copper traces is less than 1 ohm, such as about 0.4 Ohms. Accordingly, different from RTDs, small absolute variations in the fabrication process affecting the resistance of the copper traces may significantly affect the temperature measurement accuracy. Therefore, there is a need to compensate for this production inaccuracy. Options may include trimming or calibration. Trimming is cost-prohibitive at best and requires access to trim the resistance. Consequently, there is a need for thermal calibration of PCB-based sensors in DMF systems, devices, and/or cartridges.

### SUMMARY

In an aspect, described herein are methods for thermal calibration of a region of a device, comprising: (a) applying a first sense current through a sense trace of the device and a known resistance in a system holding the device; (b) determining a first set of resistance values of the sense trace and the known resistance; (c) applying a second sense current that is different than the first sense current through the sense trace and the known resistance; (d) determining a second set of resistance values of the sense trace and the known resistance; (e) measuring a set of reference temperatures near the device in the system; (f) generating calibration data correlating the first set and the second set of resistance values of the sense trace to the set of reference temperatures; and (g) reporting the calibration data for the sense trace, wherein the reporting comprises an accuracy of one or more temperature measurements associated with the region of the device. In some embodiments, the thermal calibration occurs at about 20 degrees Celsius.

In some embodiments, the method comprises adjusting a temperature of the region of the device based on the calibration data. In some embodiments, the temperature of the region of the device is adjusted to a polymerase chain reaction (PCR) denaturation temperature or a PCR annealing temperature.

In some embodiments, the device comprises a digital microfluidic cartridge configured to perform one or more droplet operations on one or more fluid droplets. In some embodiments, the one or more fluid droplets comprise biological samples, reagents, or beads. In some embodiments, the one or more fluid droplets are in contact with or substantially surrounded by an immiscible filler fluid.

In some embodiments, the one or more droplet operations comprise one or more thermocycles of heating or cooling of the one or more droplets. In some embodiments, the one or more thermocycles of heating or cooling occur at one or more rates. In some embodiments, the one or more thermocycles of heating or cooling are associated with one or more polymerase chain reactions (PCR).

In some embodiments, one or more heating elements are configured as an integrated heater to change one or more temperatures of the one or more droplets during the one or more thermocycles of heating or cooling. In some embodiments, the one or more heating elements are configured in a feedback loop with the sense trace to simultaneously adjust the one or more temperatures to within about 5% or less of a desired temperature of the one or more droplets. In some embodiments, the one or more heating elements are configured in a feedback loop with the sense trace to simultaneously adjust the one or more temperatures to within about 2% or less of a desired temperature of the one or more droplets. In some embodiments, the one or more heating elements are configured in a feedback loop with the sense trace to simultaneously adjust the one or more temperatures to within about 1% or less of a desired temperature of the one or more droplets.

In some embodiments, the sense trace is configured as an integrated sensor to measure the one or more temperatures in real-time of the one or more thermocycles of heating or cooling of the one or more droplets. In some embodiments, the sense trace comprises a thin film metal electrically connected in series with the known resistance.

In some embodiments, the sense trace comprises one or more resistive regions associated with the first set of resistance values or the second set of resistance values. In some embodiments, the first set of resistance values or the second set of resistance values comprise a resistance of at most about 1 ohm. In some embodiments, the one or more resistive regions of the sense trace are adjacent to one or more heating elements. In some embodiments, the one or more resistive regions of the sense trace comprise at least about 1 or more resistive regions associated with the first set of resistance values or the second set of resistance values. In some embodiments, the one or more resistive regions of the sense trace comprise at least about 2 or more resistive regions associated with the first set of resistance values or the second set of resistance values. In some embodiments, the one or more resistive regions of the sense trace comprise at least about 3 or more resistive regions associated with the first set of resistance values or the second set of resistance values. In some embodiments, the one or more resistive regions of the sense trace comprise about 9 resistive regions associated with the first set of resistance values or the second set of resistance values.

In some embodiments, the one or more resistive regions are spatially separated in a linear configuration. In some embodiments, the one or more resistive regions are spatially separated in an array configuration.

In some embodiments, the device is associated with an analog to digital converter (ADC) and a multiplexer (MUX) configured to record a first set of differential voltage signals associated with the first set of resistance values and the sense current. In some embodiments, the ADC and MUX are configured to record a second set of differential voltage signals associated with the second set of resistance values and the sense current. In some embodiments, the ADC and MUX are configured to record a third set of differential voltage signals associated with the known resistance and the sense current.

In some embodiments, the first sense current comprises a direct current (DC). In some embodiments, the first sense current comprises an alternating current (AC). In some embodiments, the second sense current comprises a direct current (DC). In some embodiments, the second sense current comprises an alternating current (AC).

In some embodiments, the first sense current comprises a direction that is reverse to the second sense current. In some embodiments, the first sense current comprises a magnitude at least about 0.1% or more different than the second sense current. In some embodiments, the first sense current comprises a magnitude at least about 1% or more different than the second sense current. In some embodiments, the first sense current comprises a magnitude at least about 5% or more different than the second sense current. In some embodiments, the first sense current comprises a magnitude at least about 10% or more different than the second sense current. In some embodiments, the first sense current or the second sense current comprises a phase at least about 0 degrees or more in relation to a first set of differential voltage signals associated with the first set of resistance values or a second set of differential voltage signals associated with the second set of resistance values. In some embodiments, the first sense current or the second sense current comprises a phase at least about 45 degrees or more in relation to a first set of differential voltage signals associated with the first set of resistance values or a second set of differential voltage signals associated with the second set of resistance values. In some embodiments, the first sense current or the second sense current comprises a phase at least about 90 degrees or more in relation to a first set of differential voltage signals associated with the first set of resistance values or a second set of differential voltage signals associated with the second set of resistance values.

In some embodiments, the calibration data improves the accuracy of the one or more temperature measurements by at least about 1% or more when compared to another device configured with one or more resistance temperature detectors (RTDs) having a resistance of at least about 10 or more. In some embodiments, the calibration data improves the accuracy of the one or more temperature measurements by at least about 5% or more when compared to another device configured with one or more resistance temperature detectors (RTDs) having a resistance of at least about 10 ohms or more. In some embodiments, the calibration data improves the accuracy of the one or more temperature measurements by at least about 10% or more when compared to another device configured with one or more resistance temperature detectors (RTDs) having a resistance of at least about 10 ohms or more.

In some embodiments, the calibration data comprises an accuracy within about 10% or less of a standard temperature coefficient associated with the first set of resistance values or the second set of resistance values. In some embodiments, the calibration data comprises an accuracy within about 5% or less of a standard temperature coefficient associated with the first set of resistance values or the second set of resistance values. In some embodiments, the calibration data comprises an accuracy within about 1% or less of a standard temperature coefficient associated with the first set of resistance values or the second set of resistance values.

In some embodiments, the calibration data is encoded or recorded in one or more 1D barcodes. In some embodiments, the calibration data is encoded or recorded in one or more 2D barcodes.

In some embodiments, the thermal calibration comprises a calibration time at most about 60 seconds or less. In some embodiments, the thermal calibration comprises a calibration time at most about 30 seconds or less. In some embodiments, the thermal calibration comprises a calibration time at most about 10 seconds or less.

In another aspect, described herein are systems for thermal calibration of a device, comprising: (a) a device interface configured to receive or couple the device to the system; (b) a computer system configured to control operations of the system or the device and programmed to conduct thermal calibration of a region of the device; (c) a thermal control electronics configured to control an operating temperature of the system or the device based on measurements of one or more resistance temperature detectors (RTDs); (d) one or more power sources configured to power the system or the device; and (e) a thermal calibration software configured to manage thermal calibration of the system and generate calibration data for a region of the device.

In some embodiments, the system further comprises a thermal image camera configured to provide thermal feedback of the system or the device to the computer system.

In some embodiments, the system adjusts one or more temperatures of the region of the device based on the calibration data. In some embodiments, the one or more temperatures of the region of the device are adjusted to a polymerase chain reaction (PCR) denaturation temperature or a PCR annealing temperature.

In some embodiments, the device comprises a digital microfluidic cartridge configured to perform one or more droplet operations on one or more fluid droplets. In some embodiments, the device comprises one or more heating elements configured as an integrated heater to change the one or more temperatures of the region of the device. In some embodiments, the device comprises a sense trace configured as an integrated sensor to measure the one or more temperatures of the region of the device.

In some embodiments, the calibration data is encoded or recorded in one or more 1D barcodes. In some embodiments, the calibration data is encoded or recorded in one or more 2D barcodes

In some embodiments, the thermal calibration comprises a calibration time that is at most about 60 seconds or less. In some embodiments, the thermal calibration comprises a calibration time that is at most about 30 seconds or less. In some embodiments, the thermal calibration comprises a calibration time that is at most about 10 seconds or less.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede or take precedence over any such contradictory material.

### BRIEF DESCRIPTION OF THE DRAWINGS

The features and advantages of the present disclosure will be more clearly understood from the following description taken in conjunction with the accompanying drawings, which are not necessarily drawn to scale, and wherein:

FIG. 1 illustrates a block diagram of an example of the presently disclosed DMF system that includes a DMF cartridge (or device) including integrated PCB-based heaters and PCB-based sensors and that may be used for performing thermal calibration of the DMF cartridge;

FIG. 2 illustrates a transparent perspective view of an example of a DMF instrument for holding a DMF cartridge including the integrated PCB-based heaters and PCB-based sensors and for performing thermal calibration thereof;

FIG. 3A and FIG. 3B illustrate a plan view and a cross-sectional view, respectively, of an example of one integrated PCB-based heater and its associated PCB-based sensor;

FIG. 4 illustrates is a simplified plan view of an example of a line of three integrated PCB-based heaters and showing more specific examples of sense traces;

FIG. 5 illustrates a plan view of an example of a PCB substrate including an integrated heater/sensor arrangement in relation to an electrode configuration;

FIG. 6 shows a closeup plan view of the heater/sensor arrangement of the PCB substrate shown in FIG. 5;

FIG. 7 shows a plot of the linear relationship between resistance and temperature of copper;

FIG. 8 illustrates a schematic diagram of an example of thermal control electronics of the presently disclosed DMF system for performing a thermal calibration process;

FIG. 9 through FIG. 12 illustrate schematic diagrams showing certain process operations of the presently disclosed thermal calibration process of the presently disclosed DMF system; and

FIG. 13A and FIG. 13B illustrate a flow diagram of an example of a thermal calibration process of the presently disclosed DMF system;

FIG. 14 illustrates a plan view of an example of a coded label for holding calibration data of the DMF cartridge; and

FIG. 15 illustrates a flow diagram of an example of a thermal calibration process of the presently disclosed DMF system, according to the simplest configuration.

### DEFINITIONS

“Activate,” with reference to one or more electrodes, generally means affecting a change in the electrical state of the one or more electrodes which, in the presence of a droplet, results in a droplet operation. Activation of an electrode can be accomplished using alternating current (AC) or direct current (DC). Any suitable voltage may be used.

For example, an electrode may be activated using a voltage which is greater than about 5 V, or greater than about 20 V, or greater than about 40 V, or greater than about 100 V, or greater than about 200 V or greater than about 300 V. The suitable voltage being a function of the dielectric's properties such as thickness and dielectric constant, liquid properties such as viscosity and many other factors as well. Where an AC signal is used, any suitable frequency may be employed. For example, an electrode may be activated using an AC signal having a frequency from about 1 Hz to about 10 MHz, or from about 1 Hz and 10 KHz, or from about 10 Hz to about 240 Hz, or about 60 Hz.

“cCMV” generally means congenital cytomegalovirus.

“CMV” generally means cytomegalovirus.

“Droplet” generally means a volume of liquid on a droplet actuator. In some cases, a droplet is at least partially bounded by a filler fluid. For example, a droplet may be completely surrounded by a filler fluid or may be bounded by filler fluid and one or more surfaces of the droplet actuator. As another example, a droplet may be bounded by filler fluid, one or more surfaces of the droplet actuator, and/or the atmosphere. As yet another example, a droplet may be bounded by filler fluid and the atmosphere. Droplets may, for example, be aqueous or non-aqueous or may be mixtures or emulsions including aqueous and non-aqueous components. Droplets may take a wide variety of shapes; non-limiting examples include generally disc-shaped, slug-shaped, truncated sphere, ellipsoid, spherical, partially-compressed sphere, hemispherical, ovoid, cylindrical, combinations of such shapes, and various shapes formed during droplet operations, such as merging or splitting or formed as a result of contact of such shapes with one (1) or more surfaces of a droplet actuator.

For examples of droplet fluids that may be subjected to droplet operations using the systems, devices, and methods described herein, see International Patent Application No. PCT/US 2006/047486, entitled, “Droplet-Based Biochemistry,” filed on Dec. 11, 2006, which is incorporated by reference herein in its entirety.

In various embodiments, a droplet may include a biological sample, such as whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs, and biological washes. Moreover, a droplet may include a reagent, such as water, deionized water, saline solutions, acidic solutions, basic solutions, detergent solutions, and/or buffers.

Other examples of droplet contents include reagents, such as a reagent for a biochemical protocol, such as a nucleic acid amplification protocol, an affinity-based assay protocol, an enzymatic assay protocol, a sequencing protocol, and/or a protocol for analyses of biological fluids. A droplet may include one or more beads.

“Droplet Actuator” generally means a device for manipulating droplets.

For examples of droplet actuators, see Pamula et al., U.S. Pat. No. 6,911,132, entitled “Apparatus for Manipulating Droplets by Electrowetting-Based Techniques,” issued on Jun. 28, 2005; Pamula et al., U.S. patent application Ser. No. 11/343,284, entitled “Apparatuses and Methods for Manipulating Droplets on a Printed Circuit Board,” filed on Jan. 30, 2006; Pollack et al., International Patent Application No. PCT/US2006/047486, entitled “Droplet-Based Biochemistry,” filed on Dec. 11, 2006; Shenderov, U.S. Pat. No. 6,773,566, entitled “Electrostatic Actuators for Microfluidics and Methods for Using Same,” issued on Aug. 10, 2004, and U.S. Pat. No. 6,565,727, entitled “Actuators for Microfluidics Without Moving Parts,” issued on Jan. 24, 2000; Kim and/or Shah et al., U.S. patent application Ser. No. 10/343,261, entitled “Electrowetting-driven Micropumping,” filed on Jan. 27, 2003, Ser. No. 11/275,668, entitled “Method and Apparatus for Promoting the Complete Transfer of Liquid Drops from a Nozzle,” filed on Jan. 23, 2006, Ser. No. 11/460,188, entitled “Small Object Moving on Printed Circuit Board,” filed on Jan. 23, 2006, Ser. No. 12/465,935, entitled “Method for Using Magnetic Particles in Droplet Microfluidics,” filed on May 14, 2009, and Ser. No. 12/513,157, entitled “Method and Apparatus for Real-time Feedback Control of Electrical Manipulation of Droplets on Chip,” filed on Apr. 30, 2009; Velev, U.S. Pat. No. 7,547,380, entitled “Droplet Transportation Devices and Methods Having a Fluid Surface,” issued on Jun. 16, 2009; Sterling et al., U.S. Pat. No. 7,163,612, entitled “Method, Apparatus and Article for Microfluidic Control via Electrowetting, for Chemical, Biochemical and Biological Assays and the Like,” issued on Jan. 16, 2007; Becker and Gascoyne et al., U.S. Pat. No. 7,641,779, entitled “Method and Apparatus for Programmable fluidic Processing,” issued on Jan. 5, 2010, and U.S. Pat. No. 6,977,033, entitled “Method and Apparatus for Programmable fluidic Processing,” issued on Dec. 20, 2005; Decre et al., U.S. Pat. No. 7,328,979, entitled “System for Manipulation of a Body of Fluid,” issued on Feb. 12, 2008; Yamakawa et al., U.S. Patent Pub. No. 20060039823, entitled “Chemical Analysis Apparatus,” published on Feb. 23, 2006; Wu, International Patent Pub. No. WO/2009/003184, entitled “Digital Microfluidics Based Apparatus for Heat-exchanging Chemical Processes,” published on Dec. 31, 2008; Fouillet et al., U.S. Patent Pub. No. 20090192044, entitled “Electrode Addressing Method,” published on Jul. 30, 2009; Fouillet et al., U.S. Pat. No. 7,052,244, entitled “Device for Displacement of Small Liquid Volumes Along a Micro-catenary Line by Electrostatic Forces,” issued on May 30, 2006; Marchand et al., U.S. Patent Pub. No. 20080124252, entitled “Droplet Microreactor,” published on May 29, 2008; Adachi et al., U.S. Patent Pub. No. 20090321262, entitled “Liquid Transfer Device,” published on Dec. 31, 2009; Roux et al., U.S. Patent Pub. No. 20050179746, entitled “Device for Controlling the Displacement of a Drop Between two or Several Solid Substrates,” published on Aug. 18, 2005; Dhindsa et al., “Virtual Electrowetting Channels: Electronic Liquid Transport with Continuous Channel Functionality,” Lab Chip, 10:832-836 (2010); the entire disclosures of which are incorporated by reference herein in their entirety, along with their priority documents.

Certain droplet actuators will include one or more substrates arranged with a droplet operations gap therebetween and electrodes associated with (e.g., layered on, attached to, and/or embedded in) the one (1) or more substrates and arranged to conduct one (1) or more droplet operations. For example, certain droplet actuators will include a base (or bottom) substrate, droplet operations electrodes associated with the substrate, one (1) or more dielectric layers atop the substrate and/or electrodes, and optionally one (1) or more hydrophobic layers atop the substrate, dielectric layers and/or the electrodes forming a droplet operations surface.

A top substrate may also be provided, which is separated from the droplet operations surface by a gap, commonly referred to as a “droplet operations gap.”

Various electrode arrangements on the top and/or bottom substrates are discussed in the above-referenced patents and applications and certain novel electrode arrangements are discussed in the description herein.

During droplet operations, droplets may remain in continuous contact or frequent contact with a ground or reference electrode. A ground or reference electrode may be associated with the top substrate facing the gap, the bottom substrate facing the gap, and/or in the gap. Where electrodes are provided on both substrates, electrical contacts for coupling the electrodes to a droplet actuator instrument for controlling or monitoring the electrodes may be associated with one or both plates. In some cases, electrodes on one (1) substrate are electrically coupled to the other substrate so that one (1) substrate may be in contact with the droplet actuator.

In one embodiment, a conductive material (e.g., an epoxy, such as MASTER BOND™ Polymer System EP79, available from Master Bond, Inc., Hackensack, NJ) provides the electrical connection between electrodes on one substrate and electrical paths on the other substrates, e.g., a ground electrode on a top substrate may be coupled to an electrical path on a bottom substrate by such conductive material.

Where multiple substrates are used, a spacer may be provided between the substrates to determine the height of the gap therebetween and define on-actuator dispensing reservoirs. The spacer height may, for example, be from about 5 micrometers (μm) to about 1000 μm, or about 100 μm to about 400 μm, or about 200 μm to about 350 μm, or about 250 μm to about 300 μm, or about 275 μm. The spacer may, for example, be formed of a layer of projections from the top or bottom substrates, and/or a material inserted between the top and bottom substrates.

One (1) or more openings may be provided in the one (1) or more substrates for forming a fluid path through which liquid may be delivered into the droplet operations gap. The one (1) or more openings may in some cases be aligned for interaction with one (1) or more electrodes, e.g., aligned such that liquid flowing through the opening will come into sufficient proximity with one (1) or more droplet operations electrodes to permit a droplet operation to occur by the droplet operations electrodes using the liquid.

The base (or bottom) and top substrates may in some cases be formed as one (1) integral component.

One (1) or more reference electrodes may be provided on the base (or bottom) and/or top substrates and/or in the gap. Examples of reference electrode arrangements are provided in the above-referenced patents and patent applications.

In various embodiments, the manipulation of droplets by a droplet actuator may be electrode mediated, e.g., electrowetting mediated, dielectrophoresis mediated, or Coulombic-force mediated.

Examples of other techniques for controlling droplet operations that may be used in the droplet actuators described herein include using devices that induce hydrodynamic fluidic pressure, such as those that operate on the basis of mechanical principles (e.g. external syringe pumps, pneumatic membrane pumps, vibrating membrane pumps, vacuum devices, centrifugal forces, piezoelectric/ultrasonic pumps and acoustic forces); electrical or magnetic principles (e.g. electroosmotic flow, electrokinetic pumps, ferrofluidic plugs, electrohydrodynamic pumps, attraction or repulsion using magnetic forces and magnetohydrodynamic pumps); thermodynamic principles (e.g. gas bubble generation/phase-change-induced volume expansion); other kinds of surface-wetting principles (e.g. electrowetting, and optoelectrowetting, as well as chemically, thermally, structurally and radioactively induced surface-tension gradients); gravity; surface tension (e.g., capillary action); electrostatic forces (e.g., electroosmotic flow); centrifugal flow (substrate disposed on a compact disc and rotated); magnetic forces (e.g., oscillating ions causing flow); magnetohydrodynamic forces; and vacuum or pressure differential.

In certain embodiments, combinations of two (2) or more of the foregoing techniques may be employed to conduct a droplet operation in a droplet actuator described herein. Similarly, one (1) or more of the foregoing may be used to deliver liquid into a droplet operations gap, e.g., from a reservoir in another device or from an external reservoir of the droplet actuator (e.g., a reservoir associated with a droplet actuator substrate and a flow path from the reservoir into the droplet operations gap).

Droplet operations surfaces of certain droplet actuators described herein may be made from hydrophobic materials or may be coated or treated to make them hydrophobic.

For example, in some cases, some portion or all of the droplet operations surfaces may be derivatized with low surface-energy materials or chemistries, e.g., by deposition or using in situ synthesis using compounds such as poly- or perfluorinated compounds in solution or polymerizable monomers. Examples include TEFLON® AF (available from DuPont, Wilmington, DE), members of the CYTOP® family of materials, coatings in the FLUOROPEL® family of hydrophobic and superhydrophobic coatings (available from Cytonix Corporation, Beltsville, MD), silane coatings, fluorosilane coatings, hydrophobic phosphonate derivatives (e.g. those sold by Aculon, Inc), and NOVEC™ electronic coatings (available from 3M Company, St. Paul, MN), other fluorinated monomers for plasma-enhanced chemical vapor deposition (PECVD), and organosiloxane (e.g., SiOC) for PECVD.

In some cases, the droplet operations surface may include a hydrophobic coating having a thickness ranging from about 10 nanometers (nm) to about 1,000 nm. Moreover, in some embodiments, the top substrate of the droplet actuator includes an electrically conducting organic polymer, which is then coated with a hydrophobic coating or otherwise treated to make the droplet operations surface hydrophobic. For example, the electrically conducting organic polymer deposited onto a plastic substrate may be poly(3,4-ethylenedioxythiophene) poly(styrenesulfonate) (PEDOT:PSS).

Other examples of electrically conducting organic polymers and alternative conductive layers are described in Pollack et al., International Patent Application No. PCT/US2010/040705, entitled “Droplet Actuator Devices and Methods,” the entire disclosure of which is incorporated by reference herein in its entirety.

One or both substrates may be fabricated using a printed circuit board (PCB), glass, indium tin oxide (ITO)-coated glass, and/or semiconductor materials as the substrate. When the substrate is ITO-coated glass, the ITO coating may include a thickness in the range of about 20 nm to about 200 nm, about 50 nm to about 150 nm, or about 75 nm to about 125 nm, or about 100 nm. In some cases, the top and/or bottom substrate includes a PCB substrate that is coated with a dielectric, such as a polyimide dielectric, which may in some cases also be coated or otherwise treated to make the droplet operations surface hydrophobic.

When the substrate includes a PCB, the following materials are examples of suitable materials: MITSUI™ BN-300 (available from MITSUI Chemicals America, Inc., San Jose CA); ARLON™ 11N (available from Arlon, Inc, Santa Ana, CA); NELCOR N4000-6 and N5000-30/32 (available from Park Electrochemical Corp., Melville, NY); ISOLA™ FR406 (available from Isola Group, Chandler, AZ), especially IS620; fluoropolymer family (suitable for fluorescence detection since it has low background fluorescence); polyimide family; polyester; polyethylene naphthalate; polycarbonate; polyetheretherketone; liquid crystal polymer; cyclo-olefin copolymer (COC); cyclo-olefin polymer (COP); aramid; THERMOUNT® non-woven aramid reinforcement (available from DuPont, Wilmington, DE); NOMEX® brand fiber (available from DuPont, Wilmington, DE); and paper.

Various materials are also suitable for use as the dielectric component of the substrate. Examples include: vapor deposited dielectric, such as PARYLENE™ C, PARYLENE™ N, PARYLENE™ F, and PARYLENE™ HT (for high temperature, at or about ˜300° C.) (available from Parylene Coating Services, Inc., Katy, TX); TEFLON® AF coatings; CYTOP®; soldermasks, such as liquid photoimageable soldermasks (e.g., on PCB) like TAIYO™ PSR4000 series, TAIYO™ PSR and AUS series (available from Taiyo America, Inc. Carson City, NV) (good thermal characteristics for applications involving thermal control), and PROBIMER™ 8165 (good thermal characteristics for applications involving thermal control (available from Huntsman Advanced Materials Americas Inc., Los Angeles, CA); dry film soldermask, such as those in the VACREL® dry film soldermask line (available from DuPont, Wilmington, DE); film dielectrics, such as polyimide film (e.g., KAPTON® polyimide film, available from DuPont, Wilmington, DE), polyethylene, and fluoropolymers (e.g., FEP), polytetrafluoroethylene; polyester; polyethylene naphthalate; cyclo-olefin copolymer (COC); cyclo-olefin polymer (COP); any other PCB substrate material listed above; black matrix resin; polypropylene; and black flexible circuit materials, such as DuPont Pyralux® HXC and DuPont Kapton® MBC (available from DuPont, Wilmington, DE).

Droplet transport voltage and frequency may be selected for performance with reagents used in specific assay protocols. Design parameters may be varied, e.g., number and placement of on-actuator reservoirs, number of independent electrode connections, size (volume) of different reservoirs, placement of magnets/bead washing zones, electrode size, electrode shape, inter-electrode spacing, and gap height (between the top and bottom substrates) may be varied for use with specific reagents, protocols, droplet volumes, etc.

In some cases, a substrate may be derivatized with low surface-energy materials or chemistries, e.g., using deposition or in situ synthesis using poly- or perfluorinated compounds in solution or polymerizable monomers.

Examples include TEFLON® AF coatings and FLUOROPEL® coatings for a dip or spray coating, other fluorinated monomers for plasma-enhanced chemical vapor deposition (PECVD), and organosiloxane (e.g., SiOC) for PECVD.

Additionally, in some cases, some portion or all of the droplet operations surface may be coated with a substance for reducing background noise, such as background fluorescence from a PCB substrate. For example, the noise-reducing coating may include a black matrix resin, such as the black matrix resins available from Toray Industries, Inc. (Tokyo, Japan).

Electrodes of a droplet actuator may be controlled by a controller or a processor, which is itself provided as part of a system, which may include processing functions as well as data and software storage and input and output capabilities.

Reagents may be provided on the droplet actuator in the droplet operations gap or in a reservoir fluidly coupled to the droplet operations gap. The reagents may be in liquid form, e.g., droplets, or they may be provided in a reconstitutable form in the droplet operations gap or in a reservoir fluidly coupled to the droplet operations gap. Reconstitutable reagents may be combined with liquids for reconstitution. An example of reconstitutable reagents suitable for use with the systems, devices, and methods described herein includes those described in Meathrel, et al., U.S. Pat. No. 7,727,466, entitled “Disintegratable films for diagnostic devices,” granted on Jun. 1, 2010.

“Droplet operation” generally means any manipulation of a droplet on a droplet actuator. A droplet operation may, for example, include: loading a droplet into the droplet actuator; dispensing one or more droplets from a source droplet; splitting, separating or dividing a droplet into two or more droplets; transporting a droplet from one location to another in any direction; merging or combining two or more droplets into a single droplet; diluting a droplet; mixing a droplet; agitating a droplet; deforming a droplet; retaining a droplet in position; incubating a droplet; heating a droplet; vaporizing a droplet; cooling a droplet; disposing of a droplet; transporting a droplet out of a droplet actuator; other droplet operations described herein; and/or any combination of the foregoing.

The terms “merge,” “merging,” “combine,” “combining” and the like are used to describe the creation of one droplet from two or more droplets. It can be understood that when such a term is used in reference to two or more droplets, any combination of droplet operations that are sufficient to result in the combination of the two or more droplets into one droplet may be used. For example, “merging droplet A with droplet B,” can be achieved by transporting droplet A into contact with a stationary droplet B, transporting droplet B into contact with a stationary droplet A, or transporting droplets A and B into contact with each other.

The terms “splitting,” “separating” and “dividing” and the like are used to describe the creation of more than one droplet from at least one or more droplets. These terms are not intended to imply any particular outcome with respect to the volume of the resulting droplets (e.g., the volume of the resulting droplets can be the same or different) or the number of resulting droplets (the number of resulting droplets may be 2, 3, 4, 5 or more).

The term “mixing” refers to droplet operations that result in a more homogenous distribution of one or more components within a droplet.

Examples of “loading” droplet operations include microdialysis loading, pressure-assisted loading, robotic loading, passive loading, and pipette loading.

Droplet operations may be electrode-mediated.

In some cases, droplet operations are further facilitated by the use of hydrophilic and/or hydrophobic regions on surfaces and/or by physical obstacles. For examples of droplet operations, see the patents and patent applications cited above under the definition of “droplet actuator”.

Impedance or capacitance sensing or imaging techniques may sometimes be used to determine or confirm the outcome of a droplet operation. Examples of such techniques are described in Sturmer et al., International Patent Pub. No. WO/2008/101194, entitled “Capacitance Detection in a Droplet Actuator,” published on Aug. 21, 2008, the entire disclosure of which is incorporated by reference herein in its entirety.

Generally speaking, the sensing or imaging techniques may be used to confirm the presence or absence of a droplet at a specific electrode.

For example, the presence of a dispensed droplet at the destination electrode following a droplet dispensing operation confirms that the droplet dispensing operation was effective. Similarly, the presence of a droplet at a detection spot in an assay protocol may confirm that a previous set of droplet operations has successfully produced a droplet for detection.

Droplet transport time can be quite fast. For example, in various embodiments, transport of a droplet from one electrode to the next may be completed within about 1 sec, or about 0.1 sec, or about 0.01 sec, or about 0.001 sec.

In one embodiment, the electrode is operated in AC mode but is switched to DC mode for imaging.

It is helpful for conducting droplet operations that the footprint area of a droplet to be similar to or larger than the electrowetting area; in other words, 1×-, 2×-, and 3×-droplets are usefully controlled using 1, 2, and 3 electrodes, respectively. If the droplet footprint is greater than the number of electrodes available for conducting a droplet operation at a given time, then the difference between the droplet size and the number of electrodes may not be greater than 1; in other words, a 2× droplet is usefully controlled using one (1) electrode and a 3× droplet is usefully controlled using two (2) electrodes.

When droplets include beads, it is useful for droplet size to be equal to the number of electrodes controlling the droplet, e.g., transporting the droplet.

“Filler fluid” generally means a fluid associated with a droplet operations substrate of a droplet actuator, which fluid is sufficiently immiscible with a droplet phase to render the droplet phase subject to electrode-mediated droplet operations.

For example, the droplet operations gap of a droplet actuator may be filled with a filler fluid. The filler fluid may, for example, be or include a low-viscosity oil, such as silicone oil or hexadecane filler fluid. The filler fluid may be or include a halogenated oil, such as a fluorinated or perfluorinated oil. The filler fluid may fill the entire gap of the droplet actuator or may coat one or more surfaces of the droplet actuator. Filler fluids may be selected to improve droplet operations and/or reduce loss of reagent or target substances from droplets, improve the formation of microdroplets, reduce cross-contamination between droplets, reduce contamination of droplet actuator surfaces, reduce degradation of droplet actuator materials, etc.

For example, filler fluids may be selected for compatibility with droplet actuator materials.

As an example, fluorinated filler fluids may be usefully employed with fluorinated surface coatings. Fluorinated filler fluids are useful to reduce loss of lipophilic compounds, such as umbelliferone substrates like 6-hexadecanoylamido-4-methylumbelliferone substrates (e.g., for use in Krabbe, Niemann-Pick, or other assays); other umbelliferone substrates are described in U.S. Patent Pub. No. 20110118132, published on May 19, 2011, the entire disclosure of which is incorporated herein by reference. Examples of suitable fluorinated oils include those in the Galden line, such as Galden HT170 (bp=170° C., viscosity=1.8 cSt, density=1.77 g/cm³ at 20° C.), Galden HT200 (bp=200° C., viscosity=2.4 cSt, d=1.79 g/cm³ at 20° C.), Galden HT230 (bp=230° C., viscosity=4.4 cSt, d=1.82 g/cm³ at 20° C.) (all from Solvay Solexis); those in the Novec line, such as Novec 7500 (bp=128° C., viscosity=0.8 cSt, d=1.61 g/cm³), and Fluorinert FC-40 (bp=155° C., viscosity=1.8 cSt, d=1.85 g/cm³) and Fluorinert FC-43 (bp=174° C., viscosity=2.5 cSt, d=1.86 g/cm³) (both from 3M).

In general, the selection of perfluorinated filler fluids is based on kinematic viscosity (e.g., <7 cSt) and on boiling point (e.g., >150° C.) for use in DNA/RNA-based applications (e.g., PCR, etc.).

Filler fluids may, for example, be doped with surfactants or other additives.

For example, additives may be selected to improve droplet operations and/or reduce loss of reagent or target substances from droplets, formation of microdroplets, cross-contamination between droplets, contamination of droplet actuator surfaces, degradation of droplet actuator materials, etc.

Composition of the filler fluid, including surfactant doping, may be selected for performance with reagents used in the specific assay protocols and effective interaction or non-interaction with droplet actuator materials.

Examples of filler fluids and filler fluid formulations suitable for use with the systems, devices, and methods described herein are provided in Srinivasan et al, International Patent Pub. Nos. WO/2010/027894, entitled “Droplet Actuators, Modified Fluids and Methods,” published on Mar. 11, 2010, and WO/2009/021173, entitled “Use of Additives for Enhancing Droplet Operations,” published on Feb. 12, 2009; Sista et al., International Patent Pub. No. WO/2008/098236, entitled “Droplet Actuator Devices and Methods Employing Magnetic Beads,” published on Aug. 14, 2008; and Monroe et al., U.S. Patent Publication No. 20080283414, entitled “Electrowetting Devices,” filed on May 17, 2007; the entire disclosures of which are incorporated by reference herein in their entirety, as well as the other patents and patent applications cited herein.

Fluorinated oils may in some cases be doped with fluorinated surfactants, e.g., Zonyl FSO-100 (Sigma-Aldrich) and/or others.

“Prime editing” generally means an adaptable and specific genome editing method that directly inscribes new genetic information into a specified DNA site.

“PCR” generally means “polymerase chain reaction.”

“qPCR” generally means “quantitative polymerase chain reaction.”

“Reservoir” generally means an enclosure or partial enclosure configured for holding, storing, or supplying liquid. A droplet actuator system described herein may include on-cartridge reservoirs and/or off-cartridge reservoirs.

On-cartridge reservoirs may be (i) on-actuator reservoirs, which are reservoirs in the droplet operations gap or on the droplet operations surface; (ii) off-actuator reservoirs, which are reservoirs on the droplet actuator cartridge, but outside the droplet operations gap, and not in contact with the droplet operations surface; or (iii) hybrid reservoirs which have on-actuator regions and off-actuator regions.

An example of an off-actuator reservoir is a reservoir in the top substrate. An off-actuator reservoir may be in fluid communication with an opening or flow path arranged for flowing liquid from the off-actuator reservoir into the droplet operations gap, such as into an on-actuator reservoir.

An off-cartridge reservoir may be a reservoir that is not part of the droplet actuator cartridge at all, but which flows liquid to some portion of the droplet actuator cartridge.

For example, an off-cartridge reservoir may be part of a system or docking station to which the droplet actuator cartridge is coupled during operation. Similarly, an off-cartridge reservoir may be a reagent storage container or syringe which is used to force fluid into an on-cartridge reservoir or into a droplet operations gap.

A system using an off-cartridge reservoir may include a fluid passage mechanism whereby liquid may be transferred from the off-cartridge reservoir into an on-cartridge reservoir or into a droplet operations gap.

“Washing” with respect to washing a surface, such as a hydrophilic surface, generally means reducing the amount and/or concentration of one or more substances in contact with the surface or exposed to the surface from a droplet in contact with the surface. The reduction in the amount and/or concentration of the substance may be partial, substantially complete, or even complete. The substance may be any of a wide variety of substances; examples include target substances for further analysis, and unwanted substances, such as components of a sample, contaminants, and/or excess reagent or buffer.

The terms “top,” “bottom,” “over,” “under,” and “on” are used throughout the description with reference to the relative positions of components of the droplet actuator, such as relative positions of top and bottom substrates of the droplet actuator. It will be appreciated that in many cases the droplet actuator is functional regardless of its orientation in space

When a liquid in any form (e.g., a droplet or a continuous body, whether moving or stationary) is described as being “on”, “at”, or “over” an electrode, array, matrix or surface, such liquid can be either in direct contact with the electrode/array/matrix/surface or can be in contact with one or more layers or films that are interposed between the liquid and the electrode/array/matrix/surface. In one example, filler fluid can be considered as a dynamic film between such liquid and the electrode/array/matrix/surface.

When a droplet is described as being “on” or “loaded on” a droplet actuator, it can be understood that the droplet is arranged or configured on the droplet actuator in such a manner that facilitates using (or causes) the droplet actuator to conduct one or more droplet operations on the droplet, the droplet is arranged or configured on the droplet actuator in a manner which facilitates (or causes) sensing of a property of or a signal from the droplet, and/or the droplet has been subjected to a droplet operation on the droplet actuator.

### DETAILED DESCRIPTION

The presently disclosed subject matter now will be described more fully hereinafter with reference to the accompanying drawings, in which some, but not all embodiments of the presently disclosed subject matter are shown. Like numbers refer to like elements throughout. The presently disclosed subject matter may be embodied in many different forms and may not be construed as limited to the embodiments set forth herein; rather, these embodiments are provided so that this disclosure will satisfy applicable legal requirements. Indeed, many modifications and other embodiments of the presently disclosed subject matter set forth herein may come to mind which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

In some embodiments, the presently disclosed subject matter provides a DMF system, cartridge, and methods for thermal calibration of integrated heaters and sensors. The presently disclosed DMF system provides a DMF cartridge (or device) having integrated heating. In one example, the integrated heaters and sensors may be PCB-based heaters and PCB-based sensors integrated into a PCB substrate of a DMF cartridge (or device).

In some embodiments, the presently disclosed DMF system, cartridge, and methods may provide PCB-based sensors for monitoring the temperature at respective PCB-based heaters and wherein the PCB-based sensors may be, for example, copper sense traces of a PCB substrate.

In some embodiments, the presently disclosed DMF system, cartridge, and methods may provide thermal calibration software and/or thermal control electronics for performing a thermal calibration process of a selected DMF cartridge.

In some embodiments, the presently disclosed DMF system, cartridge, and methods may provide a thermal calibration process of a selected DMF cartridge that generally may include (a) applying a first sense current through a sense trace of the device and a known resistance in a system holding the device; (b) determining a first set of resistance values of the sense trace and the known resistance; (c) applying a second sense current that is different than the first sense current through the sense trace and the known resistance; (d) determining a second set of resistance values of the sense trace and the known resistance; (e) measuring a set of reference temperatures near the device in the system; (f) generating calibration data correlating the first set and the second set of resistance values of the sense trace to the set of reference temperatures; and (g) reporting the calibration data for the sense trace, wherein the reporting comprises an accuracy of one or more temperature measurements associated with the region of the device.

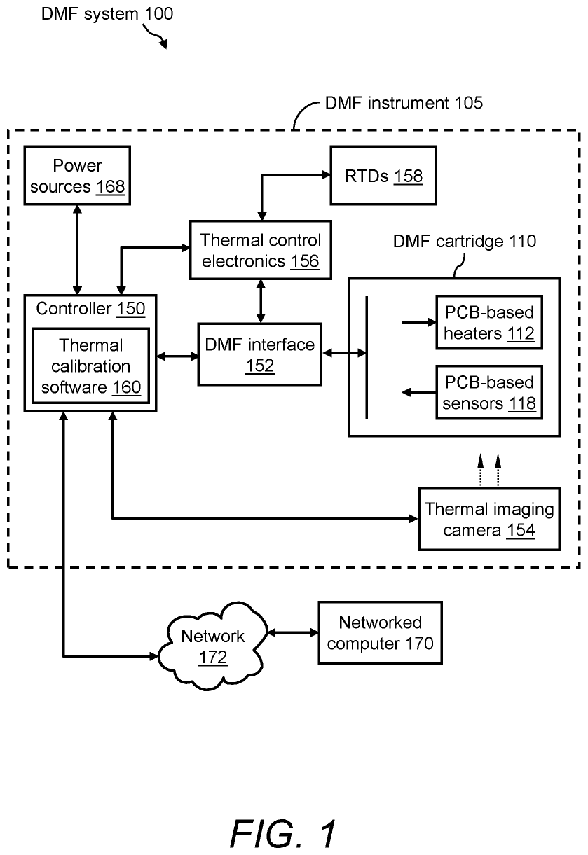

Referring now to FIG. 1 is a block diagram of an example of the presently disclosed DMF system 100 that includes a DMF cartridge (or device) 110 including integrated PCB-based heaters 112 and PCB-based sensors 118. DMF system 100 may be used for performing thermal calibration of DMF cartridge 110.

Generally, DMF system 100 provides a heating system on a DMF cartridge with electrowetting capabilities, where the heating system provides (a) closed-loop control, (b) high accuracy, (c) minimal bias, and (d) a thermal calibration process.

DMF cartridge 110 may facilitate DMF capabilities generally for fluidic actuation including droplet transporting, merging, mixing, splitting, dispensing, diluting, agitating, deforming (shaping), and other types of droplet operations. Applications of these DMF capabilities may include, for example, sample preparation and waste removal.

In one example, the DMF capabilities of DMF cartridge 110 of DMF system 100 may be used to perform assays with respect to congenital cytomegalovirus (cCMV) newborn screening.

In DMF system 100, DMF cartridge 110 may be provided, for example, as a disposable and/or reusable cartridge. Further, one or more PCB-based heaters 112 and one or more PCB-based sensors 118 may be integrated into DMF cartridge 110 in relation to the droplet operations that occur therein. Further, DMF system 100 provides closed-loop control of a heating system with high accuracy and minimal bias on DMF cartridge 110 and provides a thermal calibration process of DMF cartridge 110.

Additionally, DMF system 100 may include a controller 150, a DMF interface 152, a thermal imaging camera 154, thermal control electronics 156, one or more resistance temperature detectors (RTDs) 158, and one or more power sources 168. Controller 150 may be electrically coupled to the various hardware components of DMF system 100, such as to the DMF cartridge 110, thermal imaging camera 154, thermal control electronics 156, RTDs 158, and power sources 168. In particular, controller 150 may be electrically coupled to DMF cartridge 110 via DMF interface 152, wherein the DMF interface 152 may be, for example, a pluggable interface for connecting mechanically and electrically to a DMF cartridge 110. Together, DMF cartridge 110, controller 150, DMF interface 152, thermal imaging camera 154, thermal control electronics 156, RTDs 158, and power sources 168 may comprise a DMF instrument 105.

Controller 150 may, for example, be a general-purpose computer, special purpose computer, personal computer, tablet device, smartphone, smartwatch, microprocessor, or other programmable data processing apparatus. Controller 150 may provide processing capabilities, such as storing, interpreting, and/or executing software instructions, as well as controlling the overall operations of DMF system 100. The software instructions may comprise machine-readable code stored in non-transitory memory that is accessible by controller 150 for the execution of the instructions. Controller 150 may be configured and programmed to control data and/or power aspects of these devices. For example, with respect to DMF cartridge 110, controller 150 may control droplet manipulation by activating and/or deactivating electrodes.

Generally, controller 150 may be used for any functions of the DMF system 100.

Further, controller 150 may include algorithms for controlling any arrangement of PCB-based heaters 112.

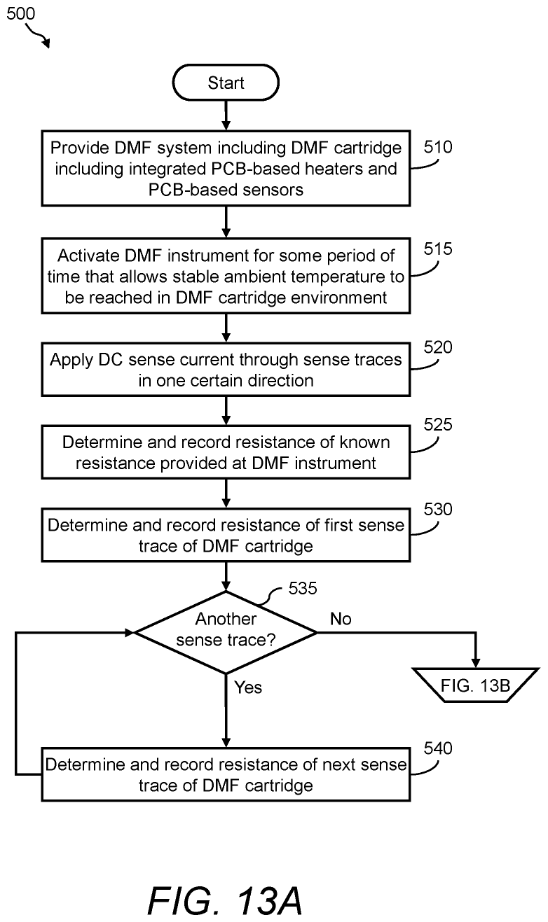

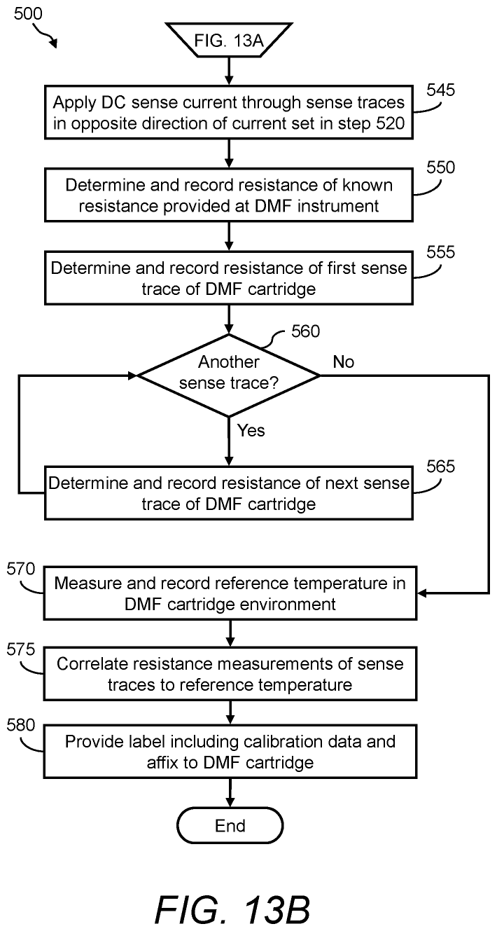

For example, controller 150 may include certain thermal calibration software 160 for managing the thermal calibration process of DMF cartridge 110 and DMF instrument 105 using the PCB-based heaters 112 and PCB-based sensors 118. More details of an example of a thermal calibration process are shown and described herein below with reference to FIG. 13A and FIG. 13B.

Optionally, DMF instrument 105 may be connected to a network. For example, controller 150 may be in communication with a networked computer 170 via a network 172. Networked computer 170 may be, for example, any centralized server or cloud server. Network 172 may be, for example, a local area network (LAN) or wide area network (WAN) for connecting to the internet.

Thermal imaging camera 154 is a type of thermographic camera that renders spatial temperature information by the measurement of infrared radiation. Thermal imaging cameras are used, for example, by firefighters to see areas of heat through smoke, darkness, or heat-permeable barriers. In DMF system 100, thermal imaging camera 154 may be, for example, the FLIR ETS320 camera available from FLIRR Systems (Sweden) or the Fluke® Ti40FT infrared camera (The Netherlands).

Thermal control electronics 156 may be provided for controlling the operating temperature of DMF cartridge 110. Thermal control electronics 156 may include, for example, any thermal sensors for controlling heaters (e.g., Peltier elements and resistive heaters) and/or coolers arranged with respect to the DMF cartridge 110. Thermal control electronics 156 may be used for interfacing with the one (1) or more PCB-based heaters 112 and one (1) or more PCB-based sensors 118 that may be integrated into DMF cartridge 110. For example, thermal control electronics 156 may provide the drive circuitry for the one (1) or more PCB-based heaters 112 and the control circuitry for the one (1) or more PCB-based sensors 118.

Additionally, thermal control electronics 156 may be used to manage the presently disclosed thermal calibration process with respect to DMF cartridge 110. Thermal control electronics 156 may be used to ensure the high accuracy of the heating system.

In one example, the heating system may include any arrangements of one (1) or more PCB-based heaters 112 and one (1) or more PCB-based sensors 118 in DMF cartridge 110 and the one (1) or more RTDs 158 in DMF instrument 105. Each of the RTDs 158 may be a passive sensor device whose resistance changes as its temperature changes. For example, the resistance increases as the temperature of the sensor increases.

Again, more details of the thermal calibration process of DMF system 100 are shown and described hereinbelow with reference to FIG. 13A and FIG. 13B.

The one (1) or more power sources 168 of DMF cartridge 110 may be, for example, one (1) or more rechargeable or non-rechargeable batteries. The one (1) or more power sources 168 supply power to any active components of DMF cartridge 110. In one example, one (1) power source 168 supplies power to the one (1) or more PCB-based heaters 112 of DMF cartridge 110 and another power source 168 supplies power to the controller 150, thermal imaging camera 154, thermal control electronics 156, and/or RTDs 158.

In DMF system 100 and with respect to PCB-based heaters 112, feedback may be used to create a “closed-loop” control system to optimize droplet actuation rate and verify droplet operations are completed successfully.

For example, PCB-based sensors 118 and thermal imaging camera 154 may be used as thermal feedback mechanisms from the PCB-based heaters 112 to controller 150 and/or thermal control electronics 156.

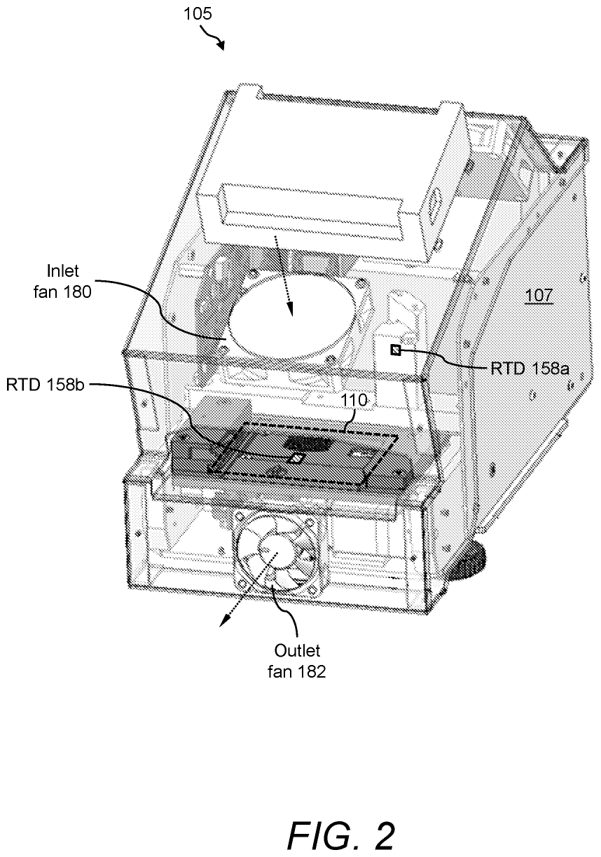

Referring now to FIG. 2 is a transparent perspective view of an example of DMF instrument 105 for holding DMF cartridge 110 including the integrated PCB-based heaters 112 and PCB-based sensors 118 and for performing thermal calibration thereof. DMF instrument 105 may include an overall instrument housing 107. With respect to thermal control and calibration of DMF cartridge 110, DMF instrument 105 may include an inlet fan 180 in the top of instrument housing 107 and an outlet fan 182 near the deck of DMF instrument 105 and wherein the deck may hold DMF cartridge 110. Accordingly, ambient air may be pulled into the top of DMF instrument 105, then across and/or past DMF cartridge 110, and then the air exits from the deck portion of DMF instrument 105.

In this example, two (2) RTDs 158 may be provided in DMF instrument 105 and in or near the airflow path therein. For example, an RTD 158a may be mounted near the air inlet (e.g., near inlet fan 180) and an RTD 158b may be mounted at the deck portion of DMF instrument 105 and near DMF cartridge 110. RTDs 158a and 160b provide feedback to controller 150 and/or thermal control electronics 156 as to the current temperature inside DMF instrument 105 and more particularly at or near DMF cartridge 110. That is, controller 150 and/or thermal control electronics 156 may be used to continuously monitor readings from RTDs 158a and 160b.

### Heating System

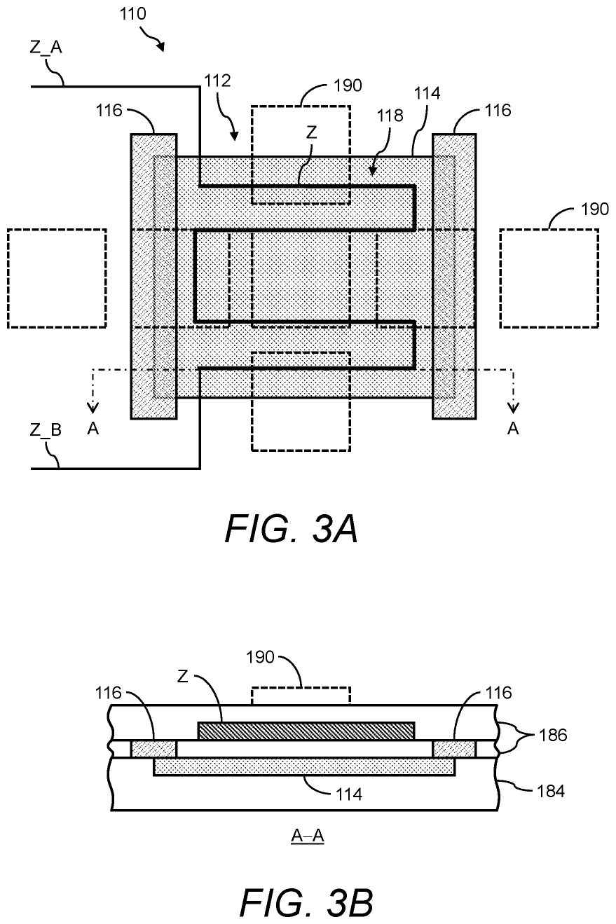

Referring now to FIG. 3A and FIG. 3B is a plan view and a cross-sectional view, respectively, of an example of one integrated PCB-based heater 112 and its associated PCB-based sensor 118. FIG. 3B is a cross-sectional view taken along line A-A of FIG. 3A. In this example, PCB-based heater 112 may include a heater element 114 arranged between two (2) heater electrical contact pads 116. Heater element 114 may be formed of resistive material, such as, but not limited to, ceramic materials such as graphite or carbon black, ceramic-metals such molybdenum disilicide, for example, metallic alloys such as nickel, molybdenum, and tungsten alloys, polymer-based materials, such as polymer thick film (PTF) heaters, or epoxy-based heaters.

In one example, heater element 114 may be a silkscreened carbon black heater element. Heater electrical contact pads 116 may be copper, gold, or silver pads. PCB-based sensor 118 may include a sense trace Z. The sense trace Z may have, for example, a serpentine path which may be contacted electrically at each end (e.g., trace contacts Z_A, Z_B). In one example, sense trace Z may be a copper sense trace Z. Additionally, FIG. 3A shows PCB-based heater 112 and its associated PCB-based sensor 118 in relation to certain lines of droplet operations electrodes 190 of DMF cartridge 110.

Referring now to FIG. 3B, PCB-based heater 112, and PCB-based sensor 118 may be formed using a multilayer PCB, such as the PCB bottom substrate of DMF cartridge 110.

For example, the structure forming the PCB-based heater 112 and the PCB-based sensor 118 may include a PCB substrate 184, a carbon-black heater element 114 formed atop the PCB substrate 184, heater electrical contact pads 116 formed atop the carbon black heater element 114, a copper sense trace Z formed atop the carbon black heater element 114, and droplet operations electrodes 190 formed atop the copper sense trace Z. Further, electrical isolation between the carbon black heater element 114, the heater electrical contact pads 116, the copper sense trace Z, and/or the droplet operations electrodes 190 may be provided by various dielectric and/or insulating layers 186.

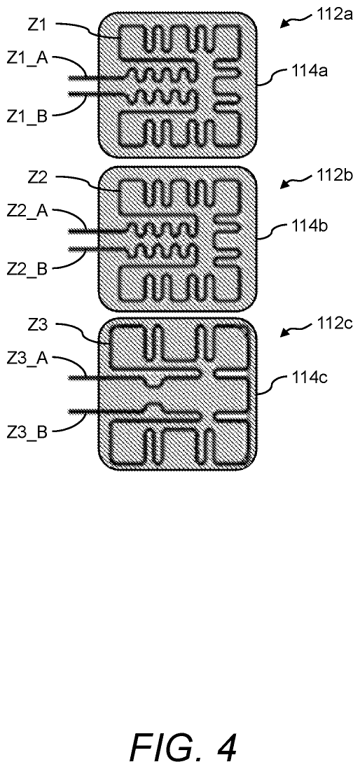

Referring now to FIG. 4 is a simplified plan view of an example of a line of three (3) integrated PCB-based heaters 112 and showing more specific examples of sense traces Z. In this example, PCB-based heaters 112a, 112b, and 112c are provided in a line. PCB-based heater 112a may include heater element 114a and a sense trace Z1 with electrical contact traces Z1_A and Z1_B. PCB-based heater 112b may include heater element 114b and a sense trace Z2 with electrical contact traces Z2_A and Z2_B. PCB-based heater 112c may include heater element 114c and a sense trace Z3 with electrical contact traces Z3_A and Z3_B.

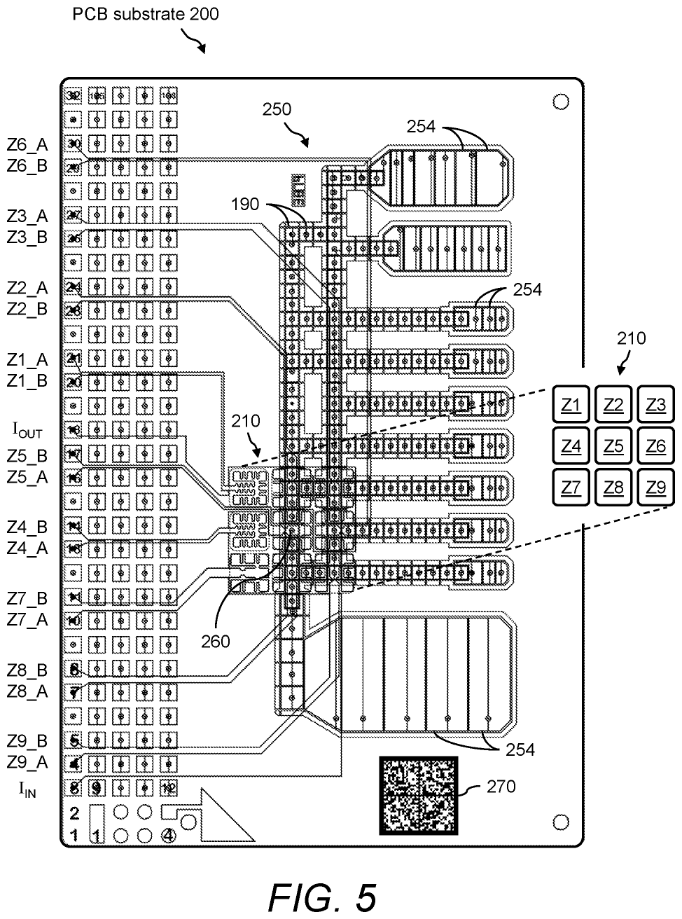

Referring now to FIG. 5 is a plan view of an example of a PCB substrate 200 including an integrated heater/sensor arrangement 210 in relation to an electrode configuration 250.

Generally, DMF devices, such as DMF cartridge 110, may consist of two (2) substrates separated by a gap that forms a chamber in which the droplet operations may be performed. In one example, a DMF device may include a silicon or printed circuit board (PCB) substrate (e.g., the bottom substrate) and a glass or plastic substrate (e.g., the top substrate) separated by the droplet operations gap. By way of example, PCB substrate 200 may be the bottom substrate of DMF cartridge 110.

In this example, heater/sensor arrangement 210 may include a 3×3 arrangement of PCB-based heaters 112 with their associated PCB-based sensors 118 in relation to a detection electrode (or spot) 260 of electrode configuration 250. Electrode configuration 250 may include, for example, various lines, paths, and/or arrays of droplet operations electrodes 190 (e.g., electrowetting electrodes).

Further, multiple arrangements of reservoir electrodes 254 supply the various lines, paths, and/or arrays of droplet operations electrodes 190. The multiple arrangements of reservoir electrodes 254 support on-cartridge reservoirs (not shown) of DMF cartridge 110. “Reservoir” generally means an enclosure or partial enclosure configured for holding, storing, and/or supplying liquid.

In FIG. 5, the 3×3 arrangement of PCB-based heaters 112 with their associated PCB-based sensors 118 are indicated by the sense traces Z1, Z2, Z3, Z4, Z5, Z6, Z7, Z8, and Z9. Further, with respect to the input/output (I/O) of PCB substrate 200, sense trace Z1 has electrical contact traces Z1_A and Z1_B, sense trace Z2 has electrical contact traces Z2_A and Z2_B, sense trace Z3 has electrical contact traces Z3_A and Z3_B, sense trace Z4 has electrical contact traces Z4 A and Z4_B, sense trace Z5 has electrical contact traces Z5_A and Z5_B, sense trace Z6 has electrical contact traces Z6_A and Z6_B, sense trace Z7 has electrical contact traces Z7_A and Z7_B, sense trace Z8 has electrical contact traces Z8_A and Z8_B, and sense trace Z9 has electrical contact traces Z9_A and Z9_B.

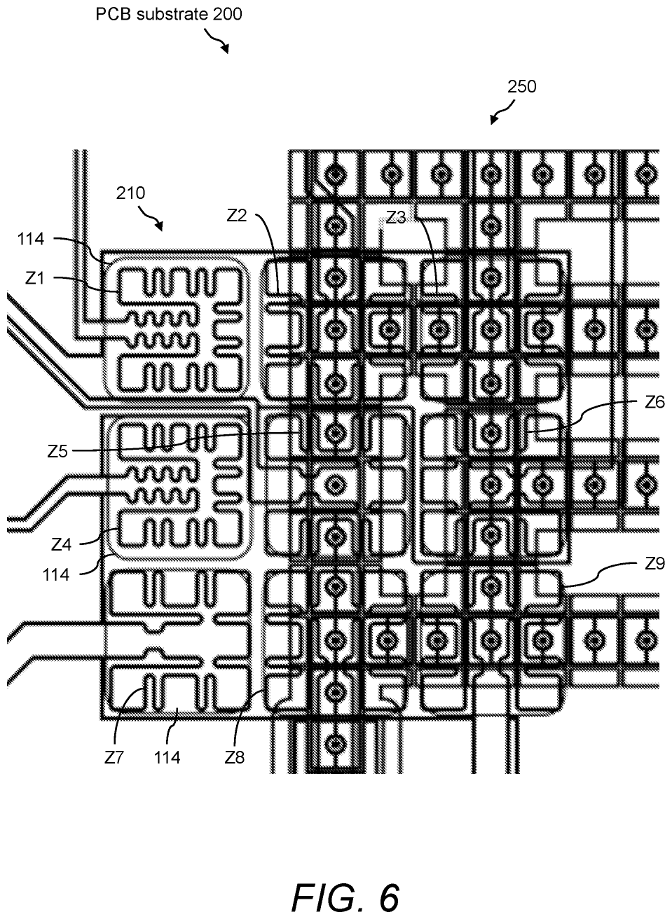

Additionally, FIG. 5 shows that PCB substrate 200 may include a current IN (I_{IN}) electrical contact and a current OUT (IOUT) electrical contact. FIG. 6 shows a close-up plan view of the heater/sensor arrangement 210 of PCB substrate 200 shown in FIG. 5.

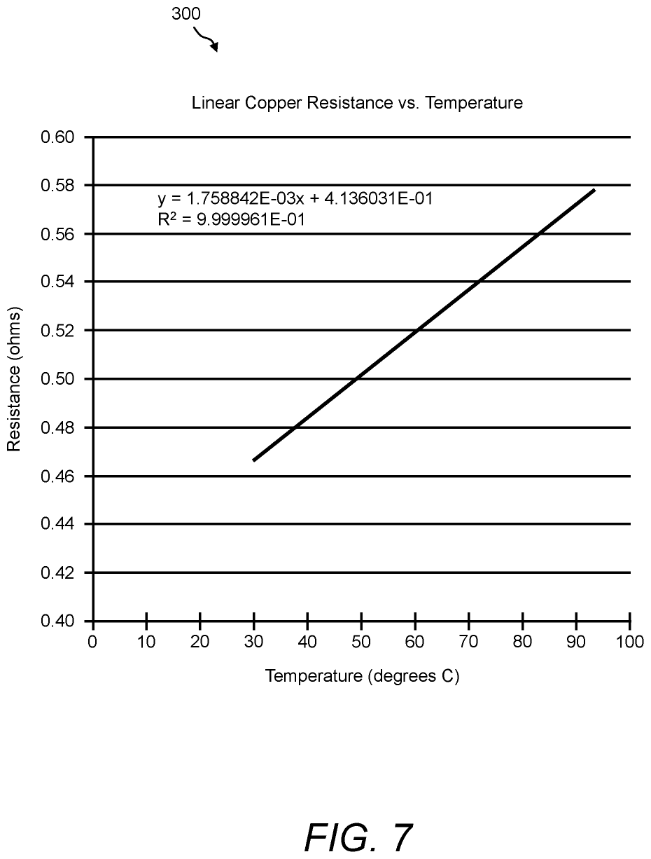

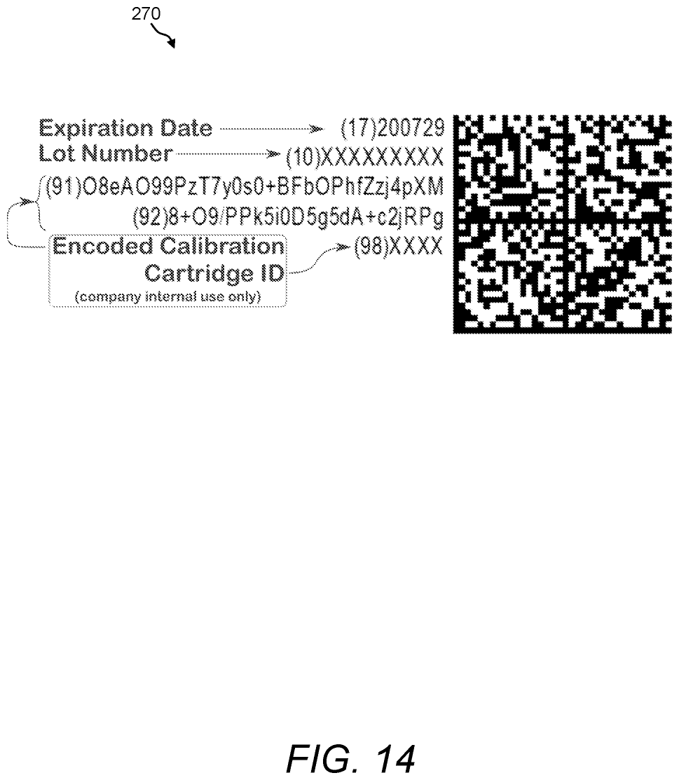

In this example, at runtime, DMF system 100 may use a real-time measure of the resistance of the nine (9) copper sense traces Z1, Z2, Z3, Z4, Z5, Z6, Z7, Z8, and Z9 on DMF cartridge 110 to infer the temperature at each of the nine (9) spatially distinct locations on DMF cartridge 110. For copper with high purity, the temperature and resistance share a linear relationship (see FIG. 7) that allows one to be determined from the other. This depends on knowing the resistance of the copper trace at a known temperature (known as the “nominal” resistance if at a temperature of 20° C.), and also on knowing the temperature coefficient of resistance (“alpha”). The fabrication of cartridges may not guarantee sufficient control of the nominal resistance of the copper traces, so it is calibrated for each cartridge. Each cartridge has the resistance of its sense traces Z measured at a controlled temperature and the resistance is referred to 20° C. for inclusion on a barcode label 270 (see FIG. 5 and FIG. 14) that is affixed to the cartridge for interrogation at runtime.

$$R = {R}_{0} * ( 1 + α * ( T - {T}_{0} ) ) \qquad Equation 1$$

$$Resistance ( R ) as a function of temperature ( T )$$

Further to the example, FIG. 7 shows plot 300 of the linear relationship between the resistance and temperature of copper, wherein the measured temperature coefficient of copper is within 1% of the accepted value.

In some embodiments, the presently disclosed DMF system 100 provides a closed, controlled loop system of sensing and heating on a DMF cartridge (e.g., DMF cartridge 110). In DMF system 100, integrating sensing on-cartridge provides real-time feedback of temperatures with high accuracy.

In each pairing of PCB-based heater 112 and PCB-based sensor 118, copper sense trace Z may be electrically connected to, for example, thermal control electronics 156 of DMF instrument 105. Thermal control electronics 156 may include a constant current source that supplies each copper sense trace Z. Thermal control electronics 156 may include data acquisition capability with respect to using PCB-based sensor 118 to measure and log the temperature at each PCB-based heater 112 using the inherent linear relationship of the resistance of copper in the sensor to the temperature of the sensor, as shown in FIG. 7.

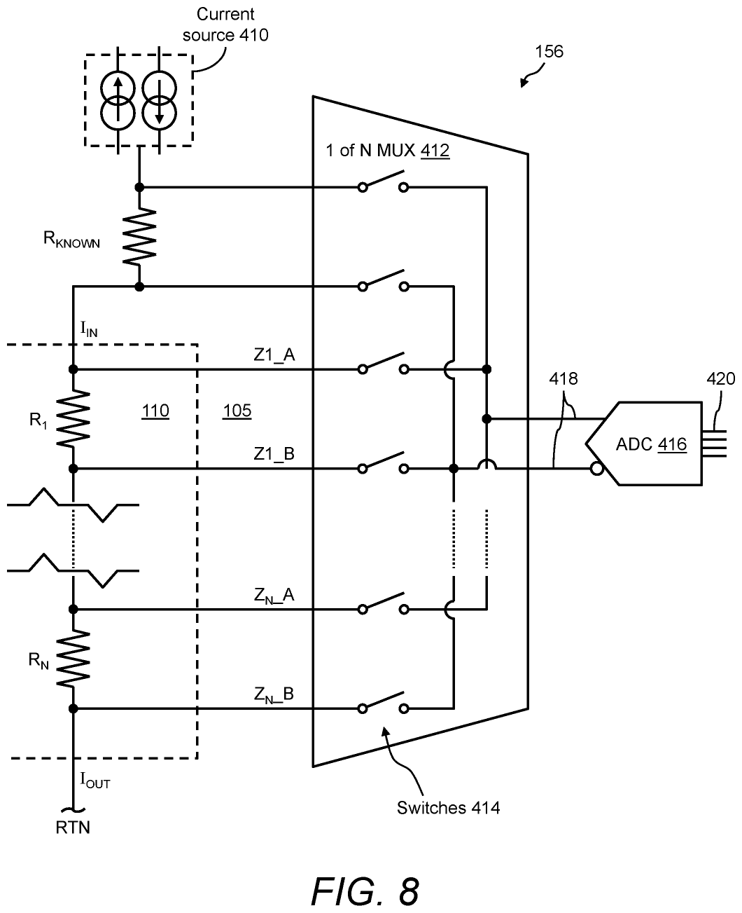

For example, FIG. 8 shows a schematic diagram of an example of thermal control electronics 156 of the presently disclosed DMF system 100 for performing a thermal calibration process. In this example, thermal control electronics 156 may include a current source 410, a 1 of N multiplexer (MUX) 412 that may include a set of analog switches 414, and an analog-to-digital converter (ADC) 416. A differential output of the 1 of N MUX 412 supplies a differential input 418 of ADC 416. ADC 416 has a set of digital outputs 420.

Current source 410 may be, for example, a constant current source and wherein the direction of the current flow may be selectable. 1 of N MUX 412 may be a standard multiplexer circuit designed to switch one of several input lines to a single common output line, albeit a differential output. In 1 of N MUX 412, the state of the analog switches 414 determines which input is directed to the output. 1 of N MUX 412 may be controlled via a set of binary select lines (not shown). ADC 416 may be a standard ADC for encoding an analog signal into a binary code. ADC 416 may be, for example, a 4-bit, 8-bit, or 16-bit ADC.

Thermal control electronics 156 may further include a known resistance, which is represented by a resistor R_{KNOWN}. Further, in DMF cartridge 110 each of the PCB-based sensors 118, such as sense traces Z1, Z2, Z3, Z4, Z5, Z6, Z7, Z8, and Z9 shown in FIG. 5, has a certain resistance, which is represented by resistors R₁ through R_{N}. In the example of the sense traces Z1, Z2, Z3, Z4, Z5, Z6, Z7, Z8, and Z9 shown in FIG. 5, DMF cartridge 110 may include resistances R₁ through R₉.

Resistor R_{KNOWN} and resistances R₁ through R₉ may be electrically connected in series and driven by current source 410. Then, the voltage across resistor R_{KNOWN} and across each of the resistances R₁ through R₉ may be measured using 1 of N MUX 412 and ADC 416. Accordingly, in this example, 1 of N MUX 412 may be at least a 1 of 10 MUX.

Because the resistance or conductivity of, for example, copper changes with temperature, thermal control electronics 156 may be used in one example to provide a constant current via current source 410 to the copper sense traces Z connected in series. Then, using 1 of N MUX 412 and ADC 416, thermal control electronics 156 may be used to monitor the voltage across the trace ends Z_A, Z_B of a selected copper sense trace Z. Subsequently, thermal control electronics 156 may be used to correlate the measured voltage value to a resistance value, and then correlate the resistance value to a temperature, wherein PCB-based heater 112 is the source of heat.

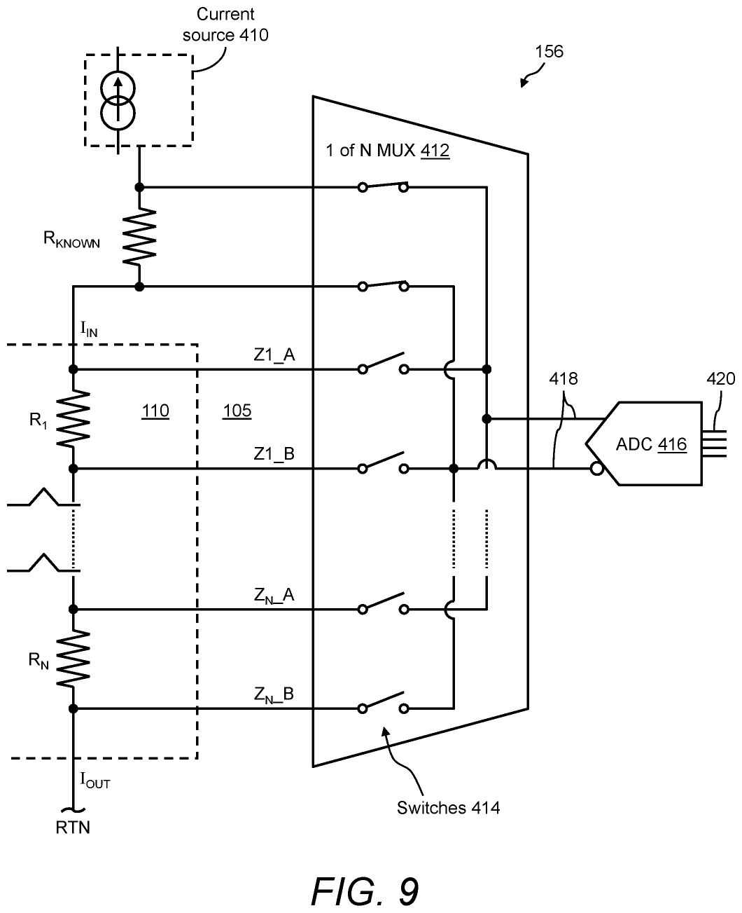

Referring now to FIG. 9 through FIG. 12 is schematic diagrams showing certain process operations of the presently disclosed thermal calibration process of DMF system 100.

In one example, FIG. 9 shows resistor R_{KNOWN} selected via 1 of N MUX 412 and current source 410 set for a certain current flow amount and direction. Then, ADC 416 may be used to measure the voltage across resistor R_{KNOWN}.

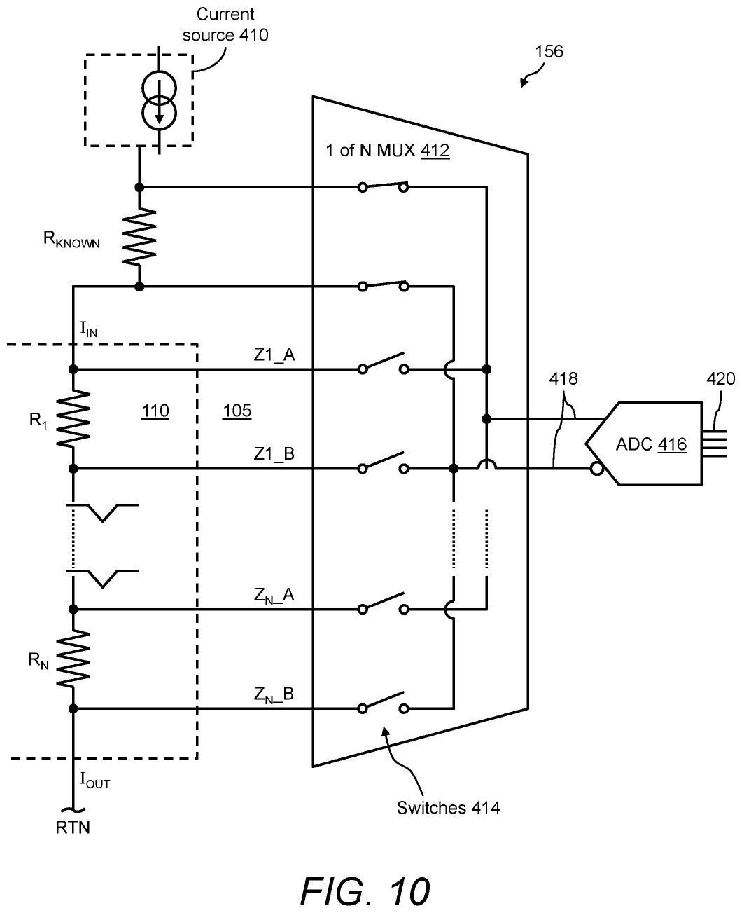

FIG. 10 shows the same scenario as shown in FIG. 9, except that the direction of the current flow may be reversed via current source 410. Then again, ADC 416 may be used to measure the voltage across resistor R_{KNOWN}. However, in DMF system 100, the current may be reversed. For example, in DMF system 100, it is not strictly necessary that the current be reversed, just that the current is something different than in the first measurement in order to address offset errors, such as “Seebeck effect” thermal voltages. A technique called “offset compensation” uses zero current instead of reversal.

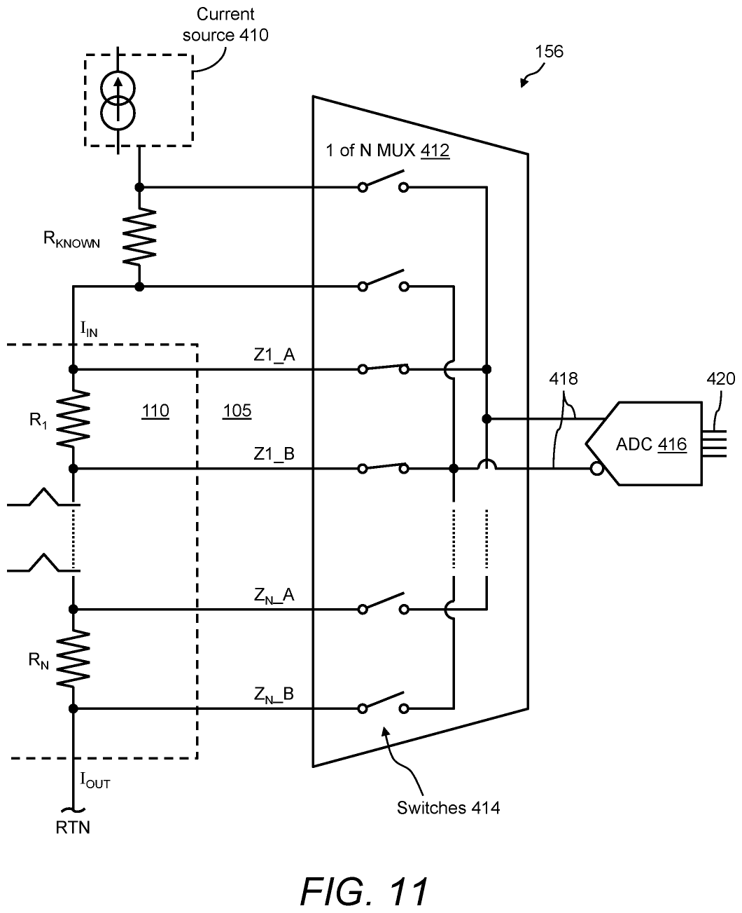

In another example, FIG. 11 shows resistor R₁ selected via 1 of N MUX 412 and current source 410 set for a certain current flow amount and direction. Then, ADC 416 may be used to measure the voltage across resistor R₁, which is sense trace Z1.

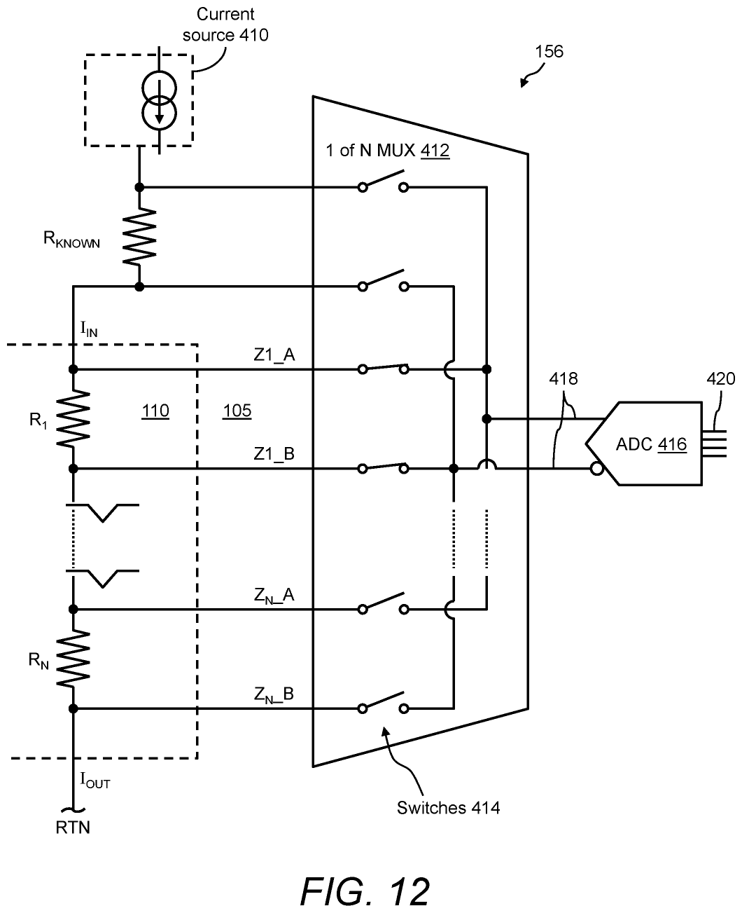

FIG. 12 shows the same scenario as shown in FIG. 11, except that the direction of the current flow is reversed via current source 410. Then again, ADC 416 may be used to measure the voltage across R₁, which is still sense trace Z1.

Referring now to FIG. 13A and FIG. 13B is a flow diagram of an example of a thermal calibration process 500 using the presently disclosed DMF system 100.

By way of example, the PCB substrate 200 shown in FIG. 5 includes nine (9) PCB-based sensors 118, which are sense traces Z1, Z2, Z3, Z4, Z5, Z6, Z7, Z8, and Z9, along with the example thermal control electronics 156 shown in FIG. 8 through FIG. 12 may be referenced in certain operations of the thermal calibration process 500. Thermal calibration process 500 may include but is not limited to, the following steps.

At operation 510, a DMF system including a DMF cartridge including integrated PCB-based heaters and PCB-based sensors is provided.

For example, DMF system 100 including DMF instrument 105 holding DMF cartridge 110, as described hereinabove with respect to FIG. 1 through FIG. 12, is provided. Further, DMF cartridge 110 includes the integrated PCB-based heaters 112 and PCB-based sensors 118 that may be used for the thermal calibration of DMF instrument 105 and DMF cartridge 110.

At operation 515, the DMF instrument is activated for some period of time that allows a stable ambient temperature to be reached in the DMF cartridge environment.

For example, DMF instrument 105 may be activated and then sit idle for from about one (1) hour to about two (2) hours, which is sufficient time for DMF cartridge 110 to reach a stable ambient temperature (e.g., substantially at or about room temperature).

In one example, FIG. 2 shows a generous ambient airflow may be directed through DMF instrument 105 and toward DMF cartridge 110 using inlet fan 180 and outlet fan 182.

In other embodiments, ovens, refrigerators, and/or closed-loop-controlled temperature baths may be used for setting the temperature in the DMF cartridge environment.

At operation 520, a DC sense current is applied through the sense traces in one (1) certain direction.

For example, using the example PCB substrate 200 shown in FIG. 5 that includes nine PCB-based sensors 118, which are sense traces Z1, Z2, Z3, Z4, Z5, Z6, Z7, Z8, and Z9, along with the example thermal control electronics 156 shown in FIG. 8 through FIG. 12, current source 410 may be used to drive a known and constant DC sense current through resistor R_{KNOWN} of DMF instrument 105 and the resistances R₁ through R₉ (e.g., representing sense traces Z1 through Z9) that are connected in series.

At operation 525, the resistance of the known resistance provided at the DMF instrument is determined and recorded.

For example, and referring now to FIG. 9, R_{KNOWN} of DMF instrument 105 may be selected using 1 of N MUX 412 and the differential voltage across R_{KNOWN} may be measured using ADC 416. Then, thermal calibration software 160 uses this measured differential voltage plus the known DC sense current from current source 410 (see FIG. 9) to calculate (using Ohm's law) the resistance of R_{KNOWN}. Then, this first resistance value of R_{KNOWN} (with current one way) is recorded.

At operation 530, the resistance of the first sense trace of the DMF cartridge is determined and recorded.

For example, and referring now to PCB substrate 200 shown in FIG. 5 and thermal control electronics 156 shown in FIG. 11, resistance R₁ (e.g., representing sense trace Z1) of DMF cartridge 110 may be selected using 1 of N MUX 412 and the differential voltage across resistance R₁ may be measured using ADC 416. Then, thermal calibration software 160 uses this measured differential voltage plus the known DC sense current from current source 410 to calculate (using Ohm's law) the resistance R₁. Then, this first value of resistance R₁ (with current one way) is recorded.

At decision operation 535, it is determined whether any other sense trace remains to be calibrated with the current in this certain direction.

In the example of sense traces Z1, Z2, Z3, Z4, Z5, Z6, Z7, Z8, and Z9 of PCB substrate 200 shown in FIG. 5, it may be determined whether any of the sense traces Z1, Z2, Z3, Z4, Z5, Z6, Z7, Z8, and Z9 remain to be processed with the current in this certain direction. If at least one (1) sense trace of the DMF cartridge 110 remains to be processed, then thermal calibration process 500 may proceed to process operation 540. However, if no sense traces of the DMF cartridge 110 remain to be processed, then thermal calibration process 500 may proceed to process operation 545.

At operation 540, the resistance of the next sense trace of the DMF cartridge is determined and recorded.

For example, and referring now to PCB substrate 200 shown in FIG. 5 and thermal control electronics 156 shown in FIG. 8, the next of the resistances R₁ through R₉ (e.g., representing sense traces Z1 through Z9) of DMF cartridge 110 may be selected using 1 of N MUX 412 and the differential voltage across the selected resistance may be measured using ADC 416. Then, thermal calibration software 160 uses this measured differential voltage plus the known DC sense current from current source 410 to calculate (using Ohm's law) the selected resistance value. Then, this first value of the selected resistance (e.g., any one of the resistances R₁ through R₉, with current one way) is recorded. Then, thermal calibration process 500 may return to process operation 535.

At operation 545, a DC sense current is applied through the sense traces in the opposite direction of the current that was set in operation 520.

For example, using the example PCB substrate 200 shown in FIG. 5 that includes nine PCB-based sensors 118, which are sense traces Z1, Z2, Z3, Z4, Z5, Z6, Z7, Z8, and Z9, along with the example thermal control electronics 156 shown in FIG. 8 through FIG. 12, current source 410 may be used to drive a known and constant DC sense current through resistor R_{KNOWN} of DMF instrument 105 and the resistances R₁ through R₉ (e.g., representing sense traces Z1 through Z9) that are connected in series. Again, in DMF system 100, the current may be reversed. For example, in this operation, it is not strictly necessary that the current be reversed, just that the current is different from the current in operation 520.

At operation 550, the resistance of the known resistance provided at the DMF instrument is determined and recorded.

For example, and referring now to FIG. 10, R_{KNOWN} of DMF instrument 105 may be selected using 1 of N MUX 412 and ADC 416 and the differential voltage across R_{KNOWN} may be measured using ADC 416. Then, thermal calibration software 160 uses this measured differential voltage plus the known DC sense current from current source 410 (see FIG. 10) to calculate (using Ohm's law) the resistance of R_{KNOWN}. Then, this second resistance value of R_{KNOWN} (with current reversed) is recorded.

At operation 555, the resistance of the first sense trace of the DMF cartridge is determined and recorded. Generally, one or more resistances R_{N} associated with one or more senses traces may be calculated according to Equation 2. In some cases, V_{NA+}-V_{NB+} represents the differential voltage associated with the resistance R_{N} when the sense current is a first sense current. V_{NA-}-V_{NB-} represents the differential voltage associated with the resistance R_{N} when the sense current is a second sense current e.g., the second sense current is reverse of the first sense current. A similar scheme may be used for the differential voltages associated with the known resistance R_{KNOWN} described as V_{KA}-V_{KB} when the sense current is the first sense current and when the sense current is the second sense current.

$${R}_{N} = {R}_{KNOWN} \times \frac{( {V}_{NA +} - {V}_{NB +} ) - ( {V}_{NA -} - {V}_{NB -} )}{( {V}_{KA +} - {V}_{KB +} ) - ( {V}_{KA -} - {V}_{KB -} )} \qquad Equation 2$$

$$Determining a resistance associated with a sense trace$$

For example, and referring now to PCB substrate 200 shown in FIG. 5 and thermal control electronics 156 shown in FIG. 11, resistance R₁ (e.g., representing sense trace Z1) of DMF cartridge 110 may be selected using 1 of N MUX 412 and the differential voltage across resistance R₁ may be measured using ADC 416. Then, thermal calibration software 160 uses this measured differential voltage plus the known DC sense current from current source 410 to calculate (using Ohm's law) the resistance R₁. Then, this second value of resistance R₁ (with current reversed) is recorded.

At a decision operation 560, it is determined whether any other sense trace remains to be calibrated with the current in the reverse direction.

In the example of sense traces Z1, Z2, Z3, Z4, Z5, Z6, Z7, Z8, and Z9 of PCB substrate 200 shown in FIG. 5, it may be determined whether any of the sense traces Z1, Z2, Z3, Z4, Z5, Z6, Z7, Z8, and Z9 remain to be processed with the current in the reverse direction. If at least one (1) sense trace of the DMF cartridge 110 remains to be processed, then thermal calibration process 500 may proceed to process operation 565. However, if no sense traces of the DMF cartridge 110 remain to be processed, then thermal calibration process 500 may proceed to process operation 570.

At operation 565, the resistance of the next sense trace of the DMF cartridge is determined and recorded.

For example, and referring now to PCB substrate 200 shown in FIG. 5 and thermal control electronics 156 shown in FIG. 8, the next of the resistances R₁ through R₉ (e.g., representing sense traces Z1 through Z9) of DMF cartridge 110 may be selected using 1 of N MUX 412 and the differential voltage across the selected resistance may be measured using ADC 416. Then, thermal calibration software 160 uses this measured differential voltage plus the known DC sense current from current source 410 to calculate (using Ohm's law) the selected resistance value. Then, this second value of the selected resistance (e.g., any one of the resistances R₁ through R₉, with current reversed) is recorded. Then, thermal calibration process 500 may return to process operation 560.

At operation 570, the reference temperature in the DMF cartridge environment is measured and recorded.

For example, the temperature of the environment of DMF cartridge 110 may be measured and recorded. More specifically, thermal calibration software 160 may be used to obtain temperature measurements from, for example, the reference RTDs 158a and 158b (see FIG. 2) of DMF instrument 105. In one example, the reference temperature may be at or about 20° C.

At operation 575, the resistance measurements of the sense traces are correlated to the reference temperature.

For example, now that thermal calibration software 160 has both a temperature measurement from the reference RTDs 158 and the measured resistances of, for example, the sense traces Z1 through Z9 of DMF cartridge 110, it can calculate the “nominal” resistances of each sense trace by referring the resistance back to 20° C. (a convention). Further, in this operation, because DMF instrument 105 does dissipate some waste power as heat and the assumption is that everything (e.g., DMF cartridge 110, DMF instrument 105, and the ambient air) are approximately at about the same temperature, a significant difference in the redundant temperature measurements will invalidate the calibration cycle and exhibit/indicate an error.

At operation 580, a label is provided that includes the calibration data and wherein the label can be affixed to the DMF cartridge.

For example, and referring now to FIG. 14, a label 270 in the form of, for example, a barcode or QR code may be provided. In this example, the calibration data may be coded into the barcode or QR code and then retrieved by scanning. In one example, the calibration data for a certain DMF cartridge 110 may be as follows:
Sense trace Z1: calibrated nominal resistance (at 20 degrees Celcius)=0.40801406 Ω
Sense trace Z2: calibrated nominal resistance (at 20° C.)=0.400422329 Ω
Sense trace Z3: calibrated nominal resistance (at 20° C.)=0.394257938 Ω
Sense trace Z4: calibrated nominal resistance (at 20° C.)=0.396707129 Ω
Sense trace Z5: calibrated nominal resistance (at 20° C.)=0.444556561 Ω
Sense trace Z6: calibrated nominal resistance (at 20° C.)=0.407826175 Ω
Sense trace Z7: calibrated nominal resistance (at 20° C.)=0.403793412 Ω
Sense trace Z8: calibrated nominal resistance (at 20° C.)=0.404919988 Ω
Sense trace Z9: calibrated nominal resistance (at 20° C.)=0.40794509 Ω

Referring now again to thermal calibration process 500 shown in FIG. 13A and FIG. 13B and thermal control electronics 156 shown in FIG. 8 through FIG. 12, resistance is defined as the slope of the voltage to current relationship of a circuit element.

In thermal calibration process 500 of DMF system 100, the lack of direct knowledge of the current, and the existence of small voltage offsets in the system complicate the measurement slightly. Additionally, the voltages are not measured directly but are converted using an analog to digital converter (e.g., ADC 416) which exhibits gain (1V is many ADC counts, or relatedly 1 ADC count may be a fraction of a volt) and practically exhibits offset errors from ideal (0V is not zero ADC counts). These issues may be addressed by measuring using two (2) different currents (e.g., process operations 520 and 545) that are indirectly measured using the known resistor R_{KNOWN} of DMF instrument 105. The gain and offset errors drop out as a result of the calculation method. This also removes constant offset errors, such as thermal offset voltages, that may cause additional offset (in addition to the ADC conversion process).

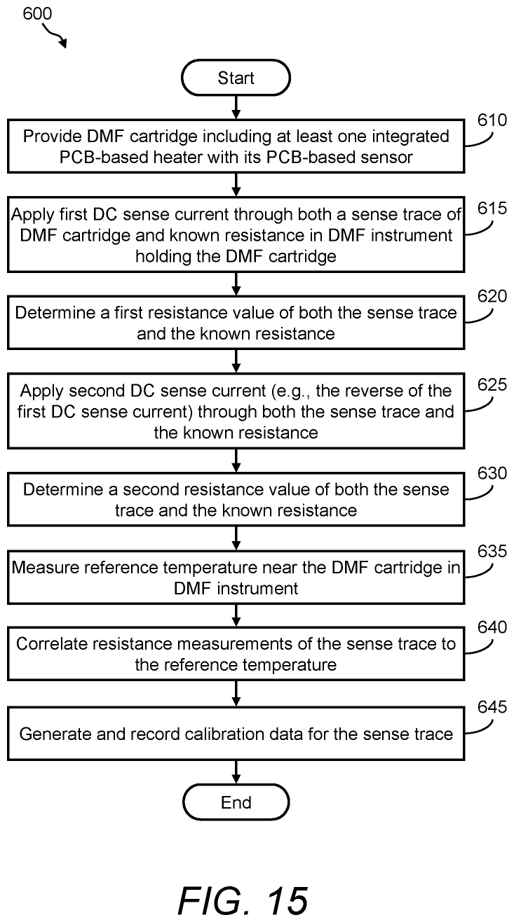

FIG. 15 illustrates a flow diagram of an example of a thermal calibration process 600 of the presently disclosed DMF system 100, according to the simplest configuration. By way of example, the PCB substrate 200 shown in FIG. 5 includes nine (9) PCB-based sensors 118, which are sense traces Z1, Z2, Z3, Z4, Z5, Z6, Z7, Z8, and Z9, along with the example thermal control electronics 156 shown in FIG. 8 through FIG. 12 may be referenced in certain operations of the thermal calibration process 600. Thermal calibration process 600 may include but is not limited to, the following operations.

At operation 610, a DMF cartridge including at least one integrated PCB-based heater with its PCB-based sensor is provided.

For example, DMF cartridge 100 including at least one (1) integrated PCB-based heater 112 with its PCB-based sensor 118 is provided. More specifically, DMF cartridge 100 including one (1) integrated PCB-based heater 112 with its copper sense trace Z1 is provided.

At operation 615, a first DC sense current is applied through both the sense trace of the DMF cartridge and a known resistance in the DMF instrument holding the DMF cartridge.

For example, under the control of thermal calibration software 160 and using current source 410 of thermal control electronics 156 (see FIG. 8), a first DC sense current may be applied through both the sense trace Z1 (e.g., resistance R₁) of the DMF cartridge 110 and resistor R_{KNOWN} of DMF instrument 105, which is holding DMF cartridge 110.

At operation 620, a first resistance value of both the sense trace and the known resistance is determined.

For example, under the control of thermal calibration software 160 and using 1 of N MUX 412 and ADC 416 of thermal control electronics 156 (see FIG. 8), a first resistance value of both the sense trace Z1 (e.g., resistance R₁) of DMF cartridge 110 and resistor R_{KNOWN} of DMF instrument 105 may be determined (e.g., calculated).

At operation 625, a second DC sense current (e.g., the reverse of the first DC sense current) is applied through both the sense trace of the DMF cartridge and the known resistance in the DMF instrument holding the DMF cartridge.

For example, under the control of thermal calibration software 160 and using current source 410 of thermal control electronics 156 (see FIG. 8), a second DC sense current (e.g., the reverse of the first DC sense current applied in process operation 615) may be applied through both the sense trace Z1 (e.g., resistance R₁) of DMF cartridge 110 and resistor R_{KNOWN} of DMF instrument 105, which is holding DMF cartridge 110. Again, in DMF system 100, the current may be reversed. For example, in this operation, it is not strictly necessary that the current be reversed, just that the current is different than in operation 615.

At operation 630, a second resistance value of both the sense trace and the known resistance is determined.

For example, under the control of thermal calibration software 160 and using 1 of N MUX 412 and ADC 416 of thermal control electronics 156 (see FIG. 8), a second resistance value of both the sense trace Z1 (e.g., resistance R₁) of DMF cartridge 110 and resistor R_{KNOWN} of DMF instrument 105 may be determined (e.g., calculated).

At operation 635, the reference temperature near the DMF cartridge in the DMF instrument is measured.

For example, the reference temperature near DMF cartridge 110 in DMF instrument 105 is measured. More specifically, thermal calibration software 160 may be used to obtain temperature measurements from, for example, the reference RTDs 158a and 158b (see FIG. 2) of DMF instrument 105. In one example, the reference temperature may be about 20° C.

At operation 640, the resistance measurements of the sense trace are correlated to the reference temperature.

For example, now that thermal calibration software 160 has both a temperature measurement from the reference RTDs 158 and the measured resistances of, for example, the sense trace Z1 of DMF cartridge 110, it can calculate the “nominal” resistance of the sense trace Z1 by referring the resistance back to 20° C. (a convention).

At operation 645, the calibration data for the sense trace is generated and recorded.

For example, and referring now again to FIG. 14, label 270 in the form of, for example, a barcode or QR code may be provided that contains the calibration data for sense trace Z1.

In another aspect, described herein are systems for thermal calibration of a device, comprising: (a) a device interface configured to receive or couple the device to the system; (b) a computer system configured to control operations of the system or the device and programmed to conduct thermal calibration of a region of the device; (c) a thermal control electronics configured to control an operating temperature of the system or the device based on measurements of one or more resistance temperature detectors (RTDs); (d) one or more power sources configured to power the system or the device; and (e) a thermal calibration software configured to manage thermal calibration of the system and generate calibration data for a region of the device.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” “comprising,” “include,” “includes,” and “including,” are intended to be non-limiting, such that recitation of items in a list is not to the exclusion of other like items that may be substituted or added to the listed items.

Terms like “preferably,” “commonly,” and “typically” are not utilized herein to limit the scope of the claimed embodiments or to imply that certain features are critical or essential to the structure or function of the claimed embodiments. These terms are intended to highlight alternative or additional features that may or may not be utilized in a particular embodiment of the present disclosure.

The term “substantially” is utilized herein to represent the inherent degree of uncertainty that may be attributed to any quantitative comparison, value, measurement, or other representation and to represent the degree by which a quantitative representation may vary from a stated reference without resulting in a change in the basic function of the present disclosure at issue.

Various modifications and variations of the disclosed methods, compositions, and uses will be apparent without departing from the scope and spirit of the present disclosure. Although the systems, devices, and methods described herein have been disclosed in connection with specific aspects or embodiments, the systems, devices, and methods described herein as claimed may not be unduly limited to such specific aspects or embodiments.

The present disclosure may be implemented using hardware, software, or a combination thereof and may be implemented in one or more computer systems or other processing systems. In one aspect, the present disclosure is directed toward one or more computer systems capable of carrying out the functionality described herein.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact but may be approximate and/or larger or smaller as desired, reflecting tolerances, conversion factors, rounding off, measurement error and the like, and other factors depending on the predetermined properties sought to be obtained by the present disclosure.

For example, the term “about,” when referring to a value can generally mean to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, can be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Unless specifically stated otherwise, terms such as “receiving,” “routing,” “updating,” “providing,” or the like, refer to actions and processes performed or implemented by computing devices that manipulates and transforms data represented as physical (electronic) quantities within the computing device's registers and memories into other data similarly represented as physical quantities within the computing device memories or registers or other such information storage, transmission or display devices. Also, the terms “first,” “second,” “third,” “fourth,” etc., as used herein generally means labels to distinguish among different elements and may not necessarily have an ordinal meaning according to their numerical designation.

Examples described herein also relate to an apparatus for performing the operations described herein. This apparatus may be specially constructed for the required purposes, or it may comprise a general-purpose computing device selectively programmed by a computer program stored in the computing device. Such a computer program may be stored in a computer-readable non-transitory storage medium.

The methods and illustrative examples described herein are not inherently related to any particular computer or other apparatus. Various general-purpose systems may be used in accordance with the teachings described herein, or it may prove convenient to construct more specialized apparatus to perform the required method operations. The required structure for a variety of these systems will appear as set forth in the description above.

It may also be noted that in some alternative implementations, the functions/acts noted may occur out of the order noted in the figures. For example, two (2) figures shown in succession may in fact be executed substantially concurrently or may sometimes be executed in the reverse order, depending upon the functionality/acts involved.

Although the method operations were described in a specific order, other operations may be performed in between described operations, described operations may be adjusted so that they occur at slightly different times, or the described operations may be distributed in a system that allows the occurrence of the processing operations at various intervals associated with the processing.

Various units, circuits, or other components may be described or claimed as “configured to” or “configurable to” perform a task or tasks. In such contexts, the phrase “configured to” or “configurable to” is used to connote structure by indicating that the units/circuits/components include structure (e.g., circuitry) that performs the task or tasks during operation. As such, the unit/circuit/component can be said to be configured to perform the task, or configurable to perform the task, even when the specified unit/circuit/component is not currently operational (e.g., is not on).

The units/circuits/components used with the “configured to” or “configurable to” language include hardware—for example, circuits, memory storing program instructions executable to implement the operation, etc. Reciting that a unit/circuit/component is “configured to” perform one or more tasks, or is “configurable to” perform one or more tasks, is expressly intended not to invoke 35 U.S.C. 112, sixth paragraph, for that unit/circuit/component.

Additionally, “configured to” or “configurable to” can include generic structure (e.g., generic circuitry) that is manipulated by software and/or firmware (e.g., an FPGA or a general-purpose processor executing software) to operate in a manner that can perform the task(s) at issue. “Configured to” may also include adapting a manufacturing process (e.g., a semiconductor fabrication facility) to fabricate devices (e.g., integrated circuits) that are adapted to implement or perform one or more tasks. “Configurable to” is expressly intended not to apply to blank media, an unprogrammed processor or unprogrammed generic computer, or an unprogrammed programmable logic device, programmable gate array, or other unprogrammed devices, unless accompanied by programmed media that confers the ability to the unprogrammed device to be configured to perform the disclosed function(s).

Although the foregoing disclosure has been described in some detail by way of illustration and example for purposes of clarity of understanding, changes and modifications can be practiced within the scope of the appended claims.

What is claimed is:

1. A method for thermal calibration of a region of a device, comprising:
(a) applying a first sense current through a sense trace of the device and a known resistance in a system holding the device;
(b) determining a first set of resistance values of the sense trace and the known resistance;
(c) applying a second sense current that is different than the first sense current through the sense trace and the known resistance;
(d) determining a second set of resistance values of the sense trace and the known resistance;
(e) measuring a set of reference temperatures near the device in the system;
(f) generating calibration data correlating the first set and the second set of resistance values of the sense trace to the set of reference temperatures; and
(g) reporting the calibration data for the sense trace, wherein the reporting comprises an accuracy of one or more temperature measurements associated with the region of the device.

2. The method of claim 1, wherein the thermal calibration occurs at about 20 degrees Celsius.

3. The method of claim 1, further comprising adjusting a temperature of the region of the device based on the calibration data.

4. The method of claim 2, wherein the temperature of the region of the device is adjusted to a polymerase chain reaction (PCR) denaturation temperature or a PCR annealing temperature.

5. The method of claim 1, wherein the device comprises a digital microfluidic cartridge configured to perform one or more droplet operations on one or more fluid droplets.

6. The method of claim 5, wherein the one or more fluid droplets comprise biological samples, reagents, or beads.

7. The method of claim 5, wherein the one or more fluid droplets are in contact with or substantially surrounded by an immiscible filler fluid.

8. The method of claim 5, wherein the one or more droplet operations comprise one or more thermocycles of heating or cooling of the one or more droplets.

9. The method of claim 8, wherein the one or more thermocycles of heating or cooling occur at one or more rates.

10. The method of claim 8, wherein the one or more thermocycles of heating or cooling are associated with one or more polymerase chain reactions (PCR).

11. The method of claim 8, wherein one or more heating elements are configured as an integrated heater to change one or more temperatures of the one or more droplets during the one or more thermocycles of heating or cooling.

12. The method of claim 11, wherein the one or more heating elements are configured in a feedback loop with the sense trace to simultaneously adjust the one or more temperatures to within about 5% or less of a desired temperature of the one or more droplets.

13. The method of claim 12, wherein the one or more heating elements are configured in a feedback loop with the sense trace to simultaneously adjust the one or more temperatures to within about 2% or less of a desired temperature of the one or more droplets.

14. The method of claim 13, wherein the one or more heating elements are configured in a feedback loop with the sense trace to simultaneously adjust the one or more temperatures to within about 1% or less of a desired temperature of the one or more droplets.

15. The method of claim 8, wherein the sense trace is configured as an integrated sensor to measure the one or more temperatures in real-time of the one or more thermocycles of heating or cooling of the one or more droplets.

16. The method of claim 15, wherein the sense trace comprises a thin film metal electrically connected in series with the known resistance.

17. The method of claim 15, wherein the sense trace comprises one or more resistive regions associated with the first set of resistance values or the second set of resistance values.

18. The method of claim 15, wherein the first set of resistance values or the second set of resistance values comprise a resistance of at most about 1 ohm.

19. The method of claim 17, wherein the one or more resistive regions of the sense trace are adjacent to one or more heating elements.

20. The method of claim 17, wherein the one or more resistive regions of the sense trace comprise at least about 1 or more resistive regions associated with the first set of resistance values or the second set of resistance values.

21. The method of claim 20, wherein the one or more resistive regions of the sense trace comprise at least about 2 or more resistive regions associated with the first set of resistance values or the second set of resistance values.

22. The method of claim 21, wherein the one or more resistive regions of the sense trace comprise at least about 3 or more resistive regions associated with the first set of resistance values or the second set of resistance values.

23. The method of claim 22, wherein the one or more resistive regions of the sense trace comprise about 9 resistive regions associated with the first set of resistance values or the second set of resistance values.

24. The method of claim 17, wherein the one or more resistive regions are spatially separated in a linear configuration.

25. The method of claim 17, wherein the one or more resistive regions are spatially separated in an array configuration.

26. The method of claim 1, wherein the device is associated with an analog to digital converter (ADC) and a multiplexer (MUX) configured to record a first set of differential voltage signals associated with the first set of resistance values and the sense current.

27. The method of claim 26, wherein the ADC and MUX are configured to record a second set of differential voltage signals associated with the second set of resistance values and the sense current.

28. The method of claim 27, wherein the ADC and MUX are configured to record a third set of differential voltage signals associated with the known resistance and the sense current.

29. The method of claim 1, wherein the first sense current comprises a direct current (DC).

30. The method of claim 1, wherein the first sense current comprises an alternating current (AC).

31. The method of claim 1, wherein the second sense current comprises a direct current (DC).

32. The method of claim 1, wherein the second sense current comprises an alternating current (AC).

33. The method of claim 1, wherein the first sense current comprises a direction that is reverse to the second sense current.

34. The method of claim 1, wherein the first sense current comprises a magnitude at least about 0.1% or more different than the second sense current.

35. The method of claim 34, wherein the first sense current comprises a magnitude at least about 1% or more different than the second sense current.

36. The method of claim 35, wherein the first sense current comprises a magnitude at least about 5% or more different than the second sense current.

37. The method of claim 36, wherein the first sense current comprises a magnitude at least about 10% or more different than the second sense current.

38. The method of claim 1, wherein the first sense current or the second sense current comprises a phase at least about 0 degrees or more in relation to a first set of differential voltage signals associated with the first set of resistance values or a second set of differential voltage signals associated with the second set of resistance values.

39. The method of claim 1, wherein the first sense current or the second sense current comprises a phase at least about 45 degrees or more in relation to a first set of differential voltage signals associated with the first set of resistance values or a second set of differential voltage signals associated with the second set of resistance values.

40. The method of claim 1, wherein the first sense current or the second sense current comprises a phase at least about 90 degrees or more in relation to a first set of differential voltage signals associated with the first set of resistance values or a second set of differential voltage signals associated with the second set of resistance values.

41. The method of claim 1, wherein the calibration data improves the accuracy of the one or more temperature measurements by at least about 1% or more when compared to another device configured with one or more resistance temperature detectors (RTDs) having a resistance of at least about 10 or more.

42. The method of claim 41, wherein the calibration data improves the accuracy of the one or more temperature measurements by at least about 5% or more when compared to another device configured with one or more resistance temperature detectors (RTDs) having a resistance of at least about 10 ohms or more.

43. The method of claim 42, wherein the calibration data improves the accuracy of the one or more temperature measurements by at least about 10% or more when compared to another device configured with one or more resistance temperature detectors (RTDs) having a resistance of at least about 10 ohms or more.

44. The method of claim 1, wherein the calibration data comprises an accuracy within about 10% or less of a standard temperature coefficient associated with the first set of resistance values or the second set of resistance values.

45. The method of claim 44, wherein the calibration data comprises an accuracy within about 5% or less of a standard temperature coefficient associated with the first set of resistance values or the second set of resistance values.

46. The method of claim 45, wherein the calibration data comprises an accuracy within about 1% or less of a standard temperature coefficient associated with the first set of resistance values or the second set of resistance values.

47. The method of claim 1, wherein the calibration data is encoded or recorded in one or more 1D barcodes.

48. The method of claim 1, wherein the calibration data is encoded or recorded in one or more 2D barcodes.

49. The method of claim 1, wherein the thermal calibration comprises a calibration time at most about 60 seconds or less.

50. The method of claim 49, wherein the thermal calibration comprises a calibration time at most about 30 seconds or less.

51. The method of claim 50, wherein the thermal calibration comprises a calibration time at most about 10 seconds or less.

52. A system for thermal calibration of a device, comprising:
(a) a device interface configured to receive or couple the device to the system;
(b) a computer system configured to control operations of the system or the device and programmed to conduct the method of claim 1;
(c) a thermal control electronics configured to control an operating temperature of the system or the device based on measurements of one or more resistance temperature detectors (RTDs);
(d) one or more power sources configured to power the system or the device; and
(e) a thermal calibration software configured to manage thermal calibration of the system and generate calibration data for a region of the device.

53. The system of claim 52, further comprising a thermal image camera configured to provide thermal feedback of the system or the device to the computer system.

54. The system of claim 52, wherein the system adjusts one or more temperatures of the region of the device based on the calibration data.

55. The system of claim 54, wherein the one or more temperatures of the region of the device are adjusted to a polymerase chain reaction (PCR) denaturation temperature or a PCR annealing temperature.

56. The system of claim 52, wherein the device comprises a digital microfluidic cartridge configured to perform one or more droplet operations on one or more fluid droplets.

57. The system of claim 56, wherein the device comprises one or more heating elements configured as an integrated heater to change the one or more temperatures of the region of the device.

58. The system of claim 57, wherein the device comprises a sense trace configured as an integrated sensor to measure the one or more temperatures of the region of the device.

59. The system of claim 52, wherein the calibration data is encoded or recorded in one or more 1D barcodes.

60. The system of claim 52, wherein the calibration data is encoded or recorded in one or more 2D barcodes

61. The system of claim 52, wherein the thermal calibration comprises a calibration time that is at most about 60 seconds or less.

62. The system of claim 61, wherein the thermal calibration comprises a calibration time that is at most about 30 seconds or less.

63. The system of claim 62, wherein the thermal calibration comprises a calibration time that is at most about 10 seconds or less.
